(19) United States
(12) Patent Application Publication
(10) Pub. No.: US 2008/0317828 A1
(43) Pub. Date: Dec. 25, 2008

(54) Fentanyl compound-containing edible patch to be applied to oral mucosa

(76) Inventors: Kazuyoshi Furusawa, Imizu-gun (JP); Kazuyoshi Sudeji, Imizu-gun (JP); Megumi Kakichi, Imizu-gun (JP); Yasunori Morimoto, Kawagoe-shi (JP); Toshinobu Seki, Sapporo-shi (JP); Yoshihiro Sawai, Imizu-gun (JP)

Correspondence Address: WENDEROTH, LIND & PONACK, L.L.P., 2033 K STREET N. W., SUITE 800, WASHINGTON, DC, 20006-1021, US

(21) Appl. No.: 12/222,944

(22) Filed: Aug. 20, 2008

Related U.S. Application Data
(62) Division of application No. 10/668,284, filed on Sep. 24, 2003.

(51) Int. Cl. A61K 9/70; A61K 31/445

(52) U.S. Cl. 424/443; 514/329

(57) ABSTRACT

There is provided a film-type edible oral mucosal patch comprising a main ingredient, a fentanyl compound, contained in a base comprising (A) a semi-synthetic water-insoluble polymer compound, (B) a semi-synthetic water-soluble polymer compound, (C) a synthetic water-soluble polymer compound, (D) a water-soluble polyhydric alcohol and (E) a pH-adjusting agent. The oral mucosal patch consists of substances accepted as the food additives or pharmaceutical additives approved for oral administration, and is industrially efficiently produced and easily and safely used.

### FIELD OF THE INVENTION

The present invention relates to a fentanyl compound-containing edible patch to be applied to oral mucosa, such a fentanyl compound being known as a drug having a high analgesic effect. More specifically, it relates to a fentanyl compound-containing mucosal patch consisting of substances accepted as food additives and/or pharmaceutical additives approved for oral administration.

### BACKGROUND OF THE INVENTION

Fentanyl compounds, in particular, fentanyl citrate, are well-known as a drug having a high analgesic effect. Administration methods are known for fentanyl compounds to be transdermally absorbed. For example, conventional transdermal absorption patches are described in Japanese Patent Laid-Open Nos. 10-45570, 2000-44476, Japanese Patent Publication Nos. 6-65347-8785 and so forth.

However, these transdermal absorption patches cause severe irritation of the application site. Therefore, these patches may be applied to the skin but should not be used on the oral mucosa since they are highly irritative to the sensitive oral mucosa. In addition, materials other than accepted food additives and pharmaceutical additives approved for oral administration (the materials not listed in the Japan's Specifications and Standards for Food Additives, Seventh Edition, and the Japanese Pharmaceutical Excipients, 1998), for example, bases such as styrene/isoprene/styrene block copolymers; adhesion imparting agents such as polyterpene resins, petroleum resins, rosin, rosin esters, oil-soluble phenol resins and N-methyl-2-pyrrolidone; and support materials such as polyethylene terephthalate, nylon, polyvinyl chloride, polyurethane, polyethylene, polyvinylidene chloride and polyamide, are used in the transdermal absorption patches. Use of such inedible materials in the oral cavity per se is a problem.

An oral administration agent for absorbing a fentanyl compound through the digestive organs is disclosed in Japanese Patent Laid-Open No. 7-206679. However, beads (acryl polymer) and an ion-exchange resin beads used therein are neither food additives nor pharmaceutical additives approved for oral administration. Oral administration of such beads into the stomach is undesirable. In addition, there is also such a problem that when a fentanyl compound is absorbed through the digestive organs, most of the compound is metabolized and disappears in the liver due to the first-pass effect. Therefore, the efficacy of the fentanyl compound may not be expected.

On the other hand, there are patent documents describing transdermal and transmucosal preparations in which a fentanyl compound is simply listed as one of the usable agents capable of being absorbed either through the skin or the mucosa. As examples of such patent documents, Japanese Patent No. 2857723, Japanese Patent Publications Nos. 4-46592 and 8-18976, Japanese Patent No. 2849937, Japanese Patent Laid-Open Nos. 6-305953 and 9-286891 may be mentioned.

However, transdermal preparations are merely described in the Examples of these patent documents, and no specific description is made for transmucosal preparations. This is also apparent from the sizes of the preparations formed in the Examples of these patent documents. These documents disclose, for example, a transdermal patch preparation punched into an oval shape of 56 mm length×45 mm wide, a transdermal patch preparation punched into a disk shape of 45 mm diameter, a heat-seal ring of 12.5 cm² concentric with an agent storage layer of 10 cm², a circular heat-seal of 0.9934 inch diameter (5 cm² microporous membrane drug-enhancer delivery surface area), an example of a cut-out of 5×5 cm in size, an example of a cut-out of 30 mm φ in size, a thin-film piece of 4×4 cm and 0.3 mm thick. From these disclosures, it is found that no consideration is given to air-passage obstruction due to accidental ingestion and thus transmucosal application, that is, intraoral application is not virtually taught.

In addition, in the same as in the above-mentioned Japanese Patent Laid-Open No. 10-45570, these preparations employ substances other than accepted food additives and pharmaceutical additives approved for oral administration, for example, an adhesion agent such as an acrylic polymer and styrene-isoprene-styrene block copolymer; and a backing layer or support material such as polyethylene terephthalate, nylon, polyvinyl chloride, polyurethane, polyethylene, polyvinylidene chloride and polyamide. Use of such inedible substances in the oral cavity where accidentally ingestion may occur is inherently a problem.

Furthermore, there are known patent documents describing a transmucosal preparation that allows the oral mucosa to absorb a drug by use of a device and a holder. Examples of such documents include Japanese Patent Laid-Open Nos. 5-500058, 5-500176 and 5-503917. These patent documents only describe a fentanyl compound as one of the drugs possibly used. Also, the device and the holder for containing a drug used in these documents are formed of materials other than accepted food additives and pharmaceutical additives approved for oral administration. Therefore, in case of accidental ingestion, air-passage obstruction may occur similarly to the above-mentioned cases.

As described above, a fentanyl compound-containing edible patch to be applied to oral mucosa, more specifically, a fentanyl compound-containing edible oral mucosal patch consisting of substances accepted as food additives or pharmaceutical additives approved for oral administration has not been specifically known.

On the other hand, not an oral mucosal patch containing a fentanyl compound but an oral mucosal patch containing a general drug for transmucosal administration is known and disclosed in many patent documents, for example, Japanese Patent Publication No. 4-4296, Japanese Patent Laid-Open Nos. 3-246220, 4-266819, 58-128314, 9-235220 and 9-194395, Japanese Patent Publication Nos. 2-60644, 5-41602 and 2001-508037, U.S. Pat. Nos. 6,159,498 and 5,800,832, etc.

Of them, there are documents concerning mucosal patches consisting of substances accepted as food additives or pharmaceutical additives approved for oral administration. However, the applicants investigated and found that these patches have the following problems.

Namely, the above-mentioned Japanese Patent Publication No. 4-4296 discloses a mucosal patch comprising a disk-shape adhesive sheet formed of a pullulan (polysaccharide) or a pullulan derivative having a high adhesive strength to the mucosa, and a control film such as ethyl cellulose film coated onto a part of the surface of the adhesive sheet (parts of one of the surfaces, the other surface, and side surface). Although the pullulan or pullulan derivative forming the adhesive sheet exhibits a high adhesive strength, the hygroscopic property is high. Therefore, there is high possibility for the adhesive sheet to adhere to a wrapping material during storage, which may cause troubles during storage and distribution. Once adhesion to the wrapping material occurs, the commercial value significantly decreases, leading to fatal results. Furthermore, ethylene glycol and diethylene glycol listed as a plasticizer to be added to the pullulan or pullulan derivative are not accepted as either food additives or pharmaceutical additives approved for oral administration.

The above-mentioned Japanese Patent Laid-Open No. 3-246220 discloses an oral mucosal adhesive film preparation having a double-layer structure comprising a mucosal adhesive layer, which is composed of a drug contained in a base of a mixture of a water-soluble cellulose derivative and a gum substance; and a mucosal non-adhesive layer, which is composed of a water-solubility decreasing agent contained in a base of a water-soluble cellulose derivative. However, this mucosal patch has a problem of delamination of the mucosal adhesive layer and the mucosal non-adhesive layer during use. Once the delamination occurs, the dissolution time of the drug layer is shortened. As a result, it is impossible to control an effective duration of a sustained release mucosal patch.

The above-mentioned Japanese Patent Laid-Open No. 4-266819 discloses a film-type oral mucosal preparation formed of a drug and a blend polymer of polyvinyl pyrrolidone polymer and (meta) acrylic copolymer. However, the mucosal patch is relatively fragile and thus a crack and/or fracture may occur. Such a fragile mucosal patch has a problem in carrying it because it crumbles in a rear pocket of a trouser. In the worst case, it is broken during manufacturing and transportation. Otherwise, a patient feels strong discomfort when it is applied to an uneven portion such as the gingiva.

The above-mentioned Japanese Patent Laid-Open No. 58-128314 discloses a mucosal preparation comprising a hydrophilic polymer layer (drug layer), which is formed of a water-soluble polymer such as carboxymethyl cellulose (CMC) or sodium polyacrylate and a plasticizer such as glycerin; and a water-insoluble or slowly-soluble polymer layer (support layer), which is formed of a water-soluble polymer such as carboxymethyl cellulose (CMC) or sodium polyacrylate, a cross-linking agent such as calcium chloride or magnesium chloride, and a plasticizer such as glycerin. In this mucosal patch, the water-insoluble or slowly-soluble polymer layer (support layer) is rendered water-insoluble due to cross-linking formed by a cross-linking agent such as calcium chloride or magnesium chloride. However, the control of the cross-linking reaction is difficult, so that it is difficult to obtain a mucosal patch with a regular quality constantly. Furthermore, collodion used to form a water-insoluble polymer layer (support layer) as described in Examples is not accepted as either a food additive or a pharmaceutical additive approved for oral administration.

The above-mentioned Japanese Patent Laid-Open No. 9-235220 discloses a multilayer film preparation comprising a drug-containing layer having a base mainly composed of a water-soluble polymer such as hydroxypropyl cellulose; a non-adhesive layer formed of hydroxypropyl cellulose, which has been rendered to be slightly soluble in water by a water-solubility decreasing agent such as shellac, stearic acid or palmitic acid and arranged on one of the surfaces of the drug-containing layer; and an adhesive layer containing a powdery adhesive substance such as carboxyvinyl polymer or pectin and arranged on the other surface of the drug-containing layer. However, it is difficult to uniformly apply the powdery adhesive substance such as a carboxyvinyl polymer or pectin to the surface of the drug-containing layer. Therefore, this mucosal patch can be formed in the laboratory level but difficult in the industrial level. Even if the preparation is industrially prepared, many steps are required, inevitably increasing manufacturing cost. Besides this, in this mucosal patch, the previously described problem, delamination, tends to occur.

The above-mentioned Japanese Patent Laid-Open No. 9-194395 discloses a pharmaceutical composition having a component containing a vinyl acetate/polyvinyl pyrrolidone copolymer and a component containing one or more compounds selected from an excipient, cellulose and a derivative thereof, starch and a derivative thereof, rubber, and so forth. This mucosal patch has a problem that the maximum amount (acceptable daily intake) of vinyl acetate to be used as a pharmaceutical additive approved in a precedent is small. Since a fentanyl compound must be administered a plurality of times per day, this mucosal patch is not suitable to be applied as a base for a rapidly-soluble mucosal patch for a fentanyl compound.

The above-mentioned Japanese Patent Publication No. 2-60644 discloses an oral mucosal patch comprising a water-soluble drug layer, which is composed of a rapidly-soluble film to be adhered to the mucosa formed of polyvinyl pyrrolidone (PVP), hydroxypropyl cellulose (HPC) and propylene glycol, and a slowly-soluble film formed of polyvinyl pyrrolidone (PVP), hydroxypropyl cellulose (HPC) and propylene glycol different in dissolution rate from the rapidly-soluble film; and a support layer, which is composed of an enteric film formed of hydroxypropylmethyl cellulose phthalate (HPMCP), triacetylene or propylene glycol, and arranged on the side of the slowly-soluble film. In this mucosal patch, the aforementioned problem, delamination, tends to occur.

The above-mentioned Japanese Patent Publication No. 5-41602 discloses, as is apparent from Examples, a halitosis-preventing agent adhesive to the oral mucosa comprising a rapidly soluble film formed of hydroxypropyl cellulose (HPC), polyvinylpyrrolidone (PVP) and macrogol 400 (polyethylene glycol 400), to which, if necessary, sodium alginate, methyl cellulose (MC) or agar may be added; and a slowly-soluble film formed of hydroxypropyl cellulose (HPC) and polyvinyl pyrrolidone (PVP), to which, if necessary, gelatin, polyvinyl alcohol, polyacrylate, carboxymethyl cellulose (CMC), starch, xanthan gum or karaya gum may be added. In this halitosis-preventing agent adhesive to the oral mucosa, the aforementioned problem, adhesion to wrapping material, tends to occur.

The above-mentioned Japanese Patent Publication No. 2001-508037, which is a patent family of U.S. Pat. Nos. 6,159,498 and 5,800,832, discloses, as is apparent from Examples, a pharmaceutical carrier device film having a multilayered structure comprising a water-soluble non-adhesive backing layer formed of hydroxyethyl cellulose, to which, if necessary, glyoxal, polyethylene glycol or propylene glycol may be added; and a water-soluble adhesive layer formed of hydroxyethyl cellulose, polyacrylate or polyvinyl pyrrolidone, and sodium carboxymethyl cellulose and to be placed in contact with the mucosa. In this film, the aforementioned problem, adhesion to wrapping material, tends to occur and crack or fracture likely occurs when the film is punched into pieces of a predetermined shape by a cutting roll.

Recently, a transmucosal patch for a narcotic analgesic agent such as a fentanyl compound has been increasingly desired in view of ease of drug administration. However, as described above, a fentanyl compound-containing oral mucosal patch consisting of substances accepted as food additives or pharmaceutical additives approved for oral administration has not yet been known. In addition, transmucosal patches for administering a general drug have many respective problems as mentioned above. Also, there are no transmucosal patches industrially (mechanically) produced and safely used. In short, there are no transmucosal patches to be applied directly to an oral mucosal patch containing a fentanyl compound.

In particular, for an oral mucosal patch containing a fentanyl compound, there are important items that should not occur during a manufacturing step, delivery/carrying step, and at the time of use.

To describe more specifically, in the manufacturing step, it is important to avoid fracture or breakage (hereinafter referred to as “punching crack”) of a mucosal patch, which occurs when a patch is punched into pieces of preparations by a cutting blade, and “delamination” which occurs when a mucosal patch is punched and a punched piece of the mucosal patch is removed from the cutting blade. If the “punching crack” and the “delamination” occur, not only the production efficiency decreases but also industrial production itself becomes difficult.

During the delivery/carrying step, the “adhesion to wrapping material” and “crack and/or fracture” mentioned above must be avoided.

When the “adhesion to wrapping material” occurs, the commercial value significantly decreases, leading to fatal results. More specifically, if a patch that likely causes the adhesion to wrapping material is introduced in the market, such a quality of the patch will prevent rapid application of the patch in emergencies. This is an important problem for an oral mucosal patch containing a fentanyl compound that is used as a narcotic analgesic agent frequently required for emergency application. In addition, under the state of the oral mucosal patch likely causing adhesion to wrapping material, it becomes difficult for fingers to remove from the patch, rendering the handling more difficult. As a result, the fingers become sticky and dirty and also the oral mucosal patch may be broken or deformed when taking it out from the wrapping.

When such a mucosal patch possibly causing the “crack and/or fracture” is carried while putting it in a rear pocket of a trouser, it may break. Therefore, the patch is unfavorable to carry. Furthermore, such a mucosal patch has drawbacks: it breaks during delivery, and a patient may feel strong discomfort when it is applied to an uneven surface such as the gingiva.

Moreover, “decomposition of a fentanyl compound” contained in the mucosal patch during a long-term storage must be avoided. Designing a mucosal patch so as not to cause “decomposition of a fentanyl compound” for attaining a long-term storage stability is a basic requirement in a preparation.

To ensure the performance of an oral mucosal patch during use, not only adhesiveness but also good permeability of a fentanyl compound of the oral mucosal patch through the oral mucosa must be attained. In other words, “poor adhesion” and “poor transmucosal permeability” must be avoided. Also, “delamination,” that is, the separation between a support layer and a drug layer in the oral cavity, must be avoided. The “delamination” may become one of causes for another problem, that is, “early-stage dissolution,” which is the phenomenon where a fentanyl compound is dissolved in a large amount in the early stage of oral administration in place of dissolving at an appropriate rate with the passage of time. The phenomenon “early-stage dissolution” of a drug must be avoided. In particular, the “early-stage dissolution” is a problem in a sustained-release oral mucosal patch.

In summary, the oral mucosal patch is required to adhere quickly to the mucosa of the oral cavity. In consideration of the adhesion site as an intraoral site, it is difficult to re-apply a patch. In other words, the patch must be applied to an application site at the first try without fail. When adhesion strength is poor (“poor adhesion”), it is difficult to adhere the patch securely to an application site at the first try, time and labor are required for application, and misattachment in position and peeling-off of the patch take place, failing in stable transmucosal absorption. Hence, “poor adhesion” strongly affects the performance of an oral mucosal patch.

If the environment of the oral cavity is not appropriately set by the oral mucosal patch so as for a fentanyl compound sufficiently to permeate through the mucosa and to be absorbed, the transmucosal permeability of a drug becomes poor (“poor transmucosal permeability”). The “poor transmucosal permeability” strongly affects the performance of an oral mucosal patch.

When the “early-stage dissolution” of a sustained-release oral mucosal patch occurs, an amount of a drug released over a period of time cannot be controlled. As the cause of the “early-stage dissolution,” it is considered that a base may flow out in an early stage, making the preparation difficult to retain its shape. It is also considered that a preparation is touched by the tongue to vary the dissolution time. Under these circumstances, it is difficult to provide a good reproducibility at the time of drug administration. Also, when the “delamination” takes place in the oral cavity, the “early-stage dissolution” takes place, causing a problem.

### SUMMARY OF THE INVENTION

The present invention has been contrived under these circumstances. An object of the present invention is to provide a fentanyl compound-containing edible oral mucosal patch consisting of substances approved as food additives and/or pharmaceutical additives approved for oral administration, which is capable of producing industrially and efficiently and overcoming all the problems such as “punching crack” and “delamination” that should not occur in a manufacturing step; “adhesion to wrapping material”, “crack and/or fracture” and “decomposition of a fentanyl compound” that should not occur in a delivery/carrying step; and “poor adhesion”, “poor transmucosal permeability”, “early-stage dissolution” and “delamination” that should not occur at the time of use.

The present inventors have conducted intensive studies to solve the aforementioned problems. As a result, they found that an a fentanyl compound-containing edible oral mucosal patch consisting of substances accepted as food additives and/or pharmaceutical additives approved for oral administration and attaining the aforementioned objects simultaneously can be obtained by organically combining the following essential ingredients: a fentanyl compound, a semi-synthetic water-insoluble polymer compound, a semi-synthetic water-soluble polymer compound, a synthetic water-soluble polymer compound, a water-soluble polyhydric alcohol and a pH-adjusting agent. The present invention was accomplished on the basis of this finding.

More specifically, the edible oral mucosal patch according to the present invention comprises a fentanyl compound, a semi-synthetic water-insoluble polymer compound, a semi-synthetic water-soluble polymer compound, a synthetic water-soluble polymer compound, a water-soluble polyhydric alcohol and a pH-adjusting agent.

The term “edible” means that the patch consists essentially of substances accepted as food additives and/or pharmaceutical additives approved for oral administration. The term semi-synthetic “water-insoluble” polymer compound means that the compound is not dissolved in purified water such as ion-exchanged water. For example, ethyl cellulose (EC) and hydroxypropylmethyl cellulose phthalate (HPMCP) are insoluble in purified water, thus belonging to the semi-synthetic water-insoluble polymer compound. Note that ethyl cellulose (EC) is insoluble in a liquid having a pH value of 5.0 or more, such as saliva; whereas, hydroxypropylmethyl cellulose phthalate (HPMCP) is dissolved.

According to a first embodiment of the present invention, the edible oral mucosal patch is formed of a single-layer.

According to a second embodiment of the present invention, the edible oral mucosal patch comprises a non-disintegrating support layer containing a semi-synthetic water-insoluble polymer compound, a synthetic water-soluble polymer compound and a water-soluble polyhydric alcohol; and a disintegrating drug layer containing a fentanyl compound, a semi-synthetic water-soluble polymer compound, a synthetic water-soluble polymer compound, a water-soluble pblyhydric alcohol and a pH-adjusting agent.

The non-disintegrating support layer, which includes a concepts such as a layer not adhere to the mucosa and a backing layer, is capable of retaining the shape of the layer at least for 5 hours after the drug layer is completely dissolved in the oral cavity. The time period of 5 hours is verified using the second fluid for the disintegration test method defined in the Japanese Pharmacopoeia. More specifically, the second fluid for the disintegration test method defined in the Japanese Pharmacopoeia is prepared by adding 118 mL of 0.2 mol/L sodium hydroxide solution and water to 250 mL of 0.2 mol/L potassium dihydrogenphosphate solution to bring a final volume of 1000 mL. The solution is clear and colorless and its pH value is about 6.8.

As another expression of the above-described “non-disintegrating support layer”, “slightly-water-soluble support layer” or “water-insoluble or slowly-water-soluble support layer” may be used. These terms have the same meaning and may be used as long as they satisfy the aforementioned conditions.

On the other hand, the disintegrating drug layer includes concepts such as a “drug-containing layer to be adhered to the mucosa”, a “drug-containing hydrophilic polymer layer” and a “drug-containing adhesive layer”, and also includes a drug layer to be dissolved in the oral cavity. The time required for dissolving the entire drug layer falls within the range from about 10 seconds to 24 hours, and more preferably, from about 10 seconds to 12 hours. The disintegration time is also determined using the second fluid for the disintegration test method of the Japanese Pharmacopoeia.

According to a third embodiment of the present invention, the edible oral mucosal patch comprises a disintegrating support layer containing a semi-synthetic water-insoluble polymer compound, a semi-synthetic water-soluble polymer compound, a water-soluble polyhydric alcohol and a pH-adjusting agent; and a disintegrating drug layer containing a fentanyl compound, a semi-synthetic water-soluble polymer compound, a synthetic water-soluble polymer compound and a water-soluble polyhydric alcohol.

The disintegrating support layer and the disintegrating drug layer refer to a support layer and a drug layer dissolved in the oral cavity. The time required for dissolving the entire support layer and the entire drug layer falls within the wide range from 10 seconds to 24 hours, and more preferably, from 10 seconds to 12 hours. The disintegration time is also determined using the second fluid for the disintegration test method of the Japanese Pharmacopoeia.

Incidentally, it is preferable to use a multilayer structure in which the disintegrating drug layer is sandwiched between the disintegrating support layers. The disintegrating support layers arranged on outer sides prevent the drug layer from adhering to a wrapping material during storage and from adhering to fingers. As a result, handling of the patch by the user can be improved. It is preferable that the dissolution-rate ratio of the disintegrating drug layer to the disintegrating support layer in the second fluid of the disintegration test method defined in the Japanese Pharmacopoeia falls within the range of 5:1 to 1:5, more preferably, 3:1 to 1:3.

Furthermore, it is preferable that the disintegrating support layer and the disintegrating drug layer are multilayered by lamination.

In the first to third embodiments of the present invention, the pH of an aqueous solution prepared by mixing 2 parts by weight of the oral mucosal patch with 98 parts by weight of water, followed by dissolving the oral mucosal patch or dissolving at least the drug layer is preferably 4.0 to 8.0.

As the fentanyl compound to be used in the edible oral mucosal patch of the present invention, fentanyl citrate is preferably used.

As the semi-synthetic water-insoluble polymer compound, at least one selected from the group consisting of ethyl cellulose (EC) and hydroxypropylmethyl cellulose phthalate (HPMCP) may be preferably used.

As the semi-synthetic water-soluble polymer compound, at least one selected from the group consisting of hydroxypropyl cellulose (HPC), hydroxypropylmethyl cellulose (HPMC), methyl cellulose (MC), sodium carboxymethyl cellulose (CMC—Na) and sodium alginate may be preferably used.

As the synthetic water-soluble polymer compound, at least one selected from the group consisting of carboxyvinyl polymer (CVP), polyvinyl pyrrolidone (PVP), polyvinyl alcohol (PVA) and sodium polyacrylate (PAA-Na) may be preferably used.

As the water-soluble polyhydric alcohol, at least one selected from the group consisting of polyethylene glycol (PEG), propylene glycol (PG), glycerin, D-sorbitol, maltitol and xylitol may be preferably used.

As the pH-adjusting agent, at least one selected from the group consisting of sodium hydroxide, sodium acetate trihydrate (CH₃COONa.3H₂O), anhydrous sodium hydrogencarbonate (sodium bicarbonate), anhydrous disodium hydrogenphosphate, disodium hydrogenphosphate dodecahydrate (Na₂HPO₄.12H₂O), trisodium phosphate dodecahydrate (Na₃PO₄.12H₂O) and calcium lactate pentahydrate (C₆H₁₀CaO₆.5H₂O) is preferably used.

### BRIEF DESCRIPTION OF THE DRAWINGS

FIG. 1 is a schematic perspective view of a measuring apparatus used in an adhesion strength test;

FIG. 2 is a graph showing the results of an in vitro transmucosal permeability test using hamster's cheek pouch with respect to preparations of Example 1 of the present invention and Comparative Example 1-E;

FIG. 3A is a schematic longitudinal sectional view of a dissolution test apparatus used in a dissolution test for oral mucosal patch;

FIG. 3B is a schematic cross-sectional view of the lower portion of the dissolution test apparatus shown in FIG. 3A;

FIG. 4 is a graph showing the dissolution test results with respect to the preparations of Example 3 of the present invention and Comparative Examples 3-1A and 3-2B;

FIG. 5 is a graph showing the results of an in vitro transmucosal permeability test using hamster's cheek pouch with respect to preparations of Example 3 of the present invention and Comparative Example 3-2E;

FIG. 6 is a schematic sectional view of the edible oral mucosal patches obtained in Examples 11 and 12 of the present invention and Comparative Example 11;

FIG. 7 is a graph showing dissolution rates of the support layer and the drug layer of the preparation of Example 11 of the present invention;

FIG. 8 is a graph showing the results of a stability test with respect to the preparations of Examples 11 and 12 of the present invention and Comparative Example 11; and

FIG. 9 is a graph showing the results of an in vitro transmucosal permeability test using hamster's cheek pouch with respect to preparations of Examples 11 and 12 of the present invention and Comparative Example 11.

### PREFERABLE EMBODIMENTS OF THE INVENTION

The edible oral mucosal patch according to the present invention is an appropriate edible film-form preparation obtained by containing a fentanyl compound in a base which comprises (A) a semi-synthetic water-insoluble polymer compound, (B) a semi-synthetic water-soluble polymer compound, (C) a synthetic water-soluble polymer compound, (D) a water-soluble polyhydric alcohol and (E) a pH-adjusting agent. More specifically, the edible oral mucosal patch is formed of a film material for adhering to the oral mucosa so as to easily administer a predetermined amount of drug; and obtained by blending five ingredients (A), (B), (C), (D) and (E), which consist of substances accepted as food additives and/or pharmaceutical additives approved for oral administration, in appropriate contents for efficient and stable absorption of a drug through the oral mucosa in a proper time, thereby adhering the patch securely onto the oral mucosa for a proper time and allowing appropriate releasing of a main ingredient, a fentanyl compound, to be effectively absorbed by the mucosa.

Furthermore, the edible oral mucosal patch of the present invention may have a multilayer structure formed of a non-disintegrating support layer comprising (A) a semi-synthetic water-insoluble polymer compound, (C) a synthetic water-soluble polymer compound and (D) a water-soluble polyhydric alcohol; and a disintegrating drug layer comprising a fentanyl compound, (B) a semi-synthetic water-soluble polymer compound, (C) a synthetic water-soluble polymer, (D) a water-soluble polyhydric alcohol and (E) a pH-adjusting agent. The multilayer structure may be punched into an appropriate size to form a film preparation. By containing an appropriate amount of a pH-adjusting agent in the disintegrating drug layer, the transmucosal absorption efficiency of a main ingredient, a fentanyl compound, can be increased.

Moreover, the edible oral mucosal patch of the present invention may have a multilayer structure formed of a disintegrating support layer comprising (A) a semi-synthetic water-insoluble polymer compound, (B) a semi-synthetic water-soluble polymer compound, (D) a water-soluble polyhydric alcohol and (E) a pH-adjusting agent; and a disintegrating drug layer comprising a fentanyl compound, (B) a semi-synthetic water-soluble polymer compound, (C) a synthetic water-soluble polymer and (D) a water-soluble polyhydric alcohol. The multilayer structure may be punched into an appropriate size to form a film preparation. By containing an appropriate amount of a pH-adjusting agent in the disintegrating support layer, the transmucosal absorption efficiency of a main ingredient, a fentanyl compound, can be increased and the stability of the fentanyl compound in the edible oral mucosal patch can be greatly improved.

When the content of the pH-adjusting agent is adjusted so as to provide pH of an aqueous solution, which is prepared by mixing 2 parts by weight of the oral mucosal patch with 98 parts by weight of water and in which at least the drug layer is dissolved, within the range of 4.0 to 8.0, more preferably, 5.0 to 7.0, a fentanyl compound can be absorbed particularly preferably. The pH of the aqueous solution herein is the pH of the aqueous solution of the drug layer obtained after about 20 to 30 g of the mixed solution containing 2 parts by weight of the oral mucosal patch and 98 parts by weight of water is shaken at 150 to 300 times/min using a shaker until it is visually confirmed that at least the drug layer is completely dissolved. More specifically, in the case of a single-layer film preparation, the pH of the aqueous solution is that of the aqueous solution obtained after the entire preparation is dissolved; in the case of a film preparation formed of a non-disintegrating support layer and a disintegrating drug layer, the pH of the aqueous solution is that of the aqueous solution obtained after the drug layer is dissolved; and in the case of a film preparation formed of a disintegrating support layer and a disintegrating drug layer, the pH of the aqueous solution is that of the aqueous solution obtained after the drug layer is dissolved. The pH of an aqueous solution may be the one of an aqueous solution obtained after the entire film preparation is completely dissolved or the one of a dispersion solution still containing an undissolved matter of the support layer. The time required for dissolving the drug layer may be controlled variously from 10 seconds to 24 hours according to the formulation. When the pH of the aqueous solution is 4.0 or less, the absorption of a fentanyl compound decreases and a sufficient flux to reach an efficient concentration in blood is not obtained. On the other hand, a pH of 8.0 or higher is not preferable since the oral mucosa may be badly affected and irritated.

Examples of the main ingredient to be used in the present invention, namely, a fentanyl compound, include fentanyl, alfentanyl, β-hydroxyfentanyl, β-hydroxy-3-methylfentanyl, ρ-fluorofentanyl, α-methylthiofentanyl, 3-methylthiofentanyl, 3-methylfentanyl, acetyl-α-methylfentanyl, α-methylfentanyl, sufentanyl, remifentanyl, lofentanyl and kerfentanyl. Of them, fentanyl is preferably used. Furthermore, salts of fentanyl, in particular, fentanyl citrate, are preferably used.

As each of the ingredients (A), (B), (C), (D) and (E) to be used in the oral mucosal patch of the present invention, it is preferable to use edible one satisfying standards such as the Japanese Pharmacopoeia, the Japanese Pharmaceutical Codex, the Japanese Pharmaceutical Excipients, the Japan's Specifications and Standards for Food Additives, the National Formulary, the United States Pharmacopeia and the like.

Of them, particularly preferable substances are as follows. As (A) a semi-synthetic water-insoluble polymer compound, ethyl cellulose (EC), hydroxypropylmethyl cellulose phthalate (HPMCP), and the like, may be used singly or in combination. The semi-synthetic water-insoluble polymer compound refers to one insoluble in purified water such as ion-exchanged water. For example, ethyl cellulose (EC) and hydroxypropylmethyl cellulose phthalate (HPMCP) are insoluble in purified water and therefore belong to the semi-synthetic water-insoluble polymer compound. Note that ethyl cellulose (EC) is insoluble in a liquid of pH 5.0 or higher such as saliva; whereas, hydroxypropylmethyl cellulose phthalate (HPMCP) is soluble.

As water-insoluble ethyl cellulose, it is preferably to use ethyl cellulose having the degree of etherification of 43 to 50%, the degree of substitution of 2.25 to 2.58, and 46.5 to 51.0% by dry weight of an ethoxyl group (—OC₂H₆). As water-insoluble hydroxypropylmethyl cellulose phthalate (HPMCP), it is preferable to use HPMCP containing 18 to 24% by dry weight of a methoxyl group (—OCH₃), 5 to 10% by dry weight of a hydroxypropoxyl group (—OC₃H₆OH), and 21 to 35% by dry weight of a carboxybenzoyl group.

As (B) a semi-synthetic water-soluble polymer compound, hydroxypropyl cellulose (HPC), hydroxypropylmethyl cellulose (HPMC), methyl cellulose (MC), sodium carboxymethyl cellulose (CMC—Na), sodium alginate, and the like may be used in single or in combination.

As water-soluble hydroxypropyl cellulose (HPC), it is preferable to use HPC having 53.4 to 77.50% by dry weight of hydroxypropoxyl group. As water-soluble hydroxypropylmethyl cellulose (HPMC), it is preferable to use HPMC having 19.0 to 30.0% by dry weight of a methoxyl group, and 4 to 12% by dry weight of hydroxypropoxyl group. As water-soluble methyl cellulose (MC), it is preferable to use MC containing 26.0 to 33.0% by dry weight of a methoxyl group. As water-soluble sodium carboxymethyl cellulose (CMC—Na), it is preferable to use CMC—Na containing 6.5 to 8.5% by dry weight of sodium. As water-soluble sodium alginate, it is preferable to use sodium alginate having a molecular weight of 40,000 to 200,000.

As (C) a synthetic water-soluble polymer compound, carboxyvinyl polymer (CVP), polyvinyl pyrrolidone (PVP), polyvinylalcohol (PVA), sodiumpolyacrylate (PAA-Na) and the like may be used singly or in combination.

As water-soluble carboxyvinyl polymer (CVP), it is preferable to use CVP containing 58.0 to 63.0% by dry weight of a carboxyl group. As water-soluble polyvinyl pyrrolidone (PVP), it is preferable to use PVP having a K value (intrinsic viscosity, also called Fikentscher's K value which is used as a means for distinguishing individual grades) of 10 to 120. As water-soluble sodium polyacrylate (PAA-Na), it is preferable to use 0.2% by weight aqueous solution of PAA-Na having a pH of 6.2 to 10.

As (D) a water-soluble polyhydric alcohol, polyethylene glycol, propylene glycohol (PG), glycerin, D-sorbitol, maltitol, xylitol and the like may be used singly or in combination.

As water-soluble polyethylene glycol, it is preferable to use polyethylene glycol having an average molecular weight of 300 to 35,000 measured by the average molecular weight test method defined in the Japanese Pharmacopoeia or the National Formulary.

As (E) a pH-adjusting agent, sodium hydroxide, sodium acetate trihydrate (CH₃COONa.3H₂O), anhydrous sodium hydrogencarbonate (sodium bicarbonate), anhydrous disodium hydrogenphosphate, disodium hydrogenphosphate dodecahydrate (Na₂HPO₄.12H₂O), trisodium phosphate dodecahydrate (Na₃PO₄.12H₂O) and calcium lactate pentahydrate (C₆H₁₀CaO₆.5H₂O) may be used singly or in combination. Note that sodium polyacrylate mentioned as the ingredient (C) may be used also as a pH-adjusting agent.

When the edible oral mucosal patch of the present invention is prepared, various additives may be added if necessary. Examples of these additives include general base materials such as chitosan, starch and pectine; binding agents such as tragacanth powder, gum arabic, cornstarch and gelatin; excipients such as microcrystalline cellulose; disintegrators such as cornstarch and α-starch; lubricants such as magnesium stearate; sweetening agents such as sucrose, lactose, saccharin and aspartame; sweetening agents or preservatives such as peppermint, peppermint oil and cherry; opaquers such as titanium oxide; and coloring agents such as red ferric oxide (diiron trioxide).

Now, a method of preparing an edible oral mucosal patch of the present invention will be described below.

### 1) Single-Layer Preparation

To an appropriate amount of purified water, (E) a pH-adjusting agent is added and dissolved while stirring. After an appropriate amount of solvent (e.g., ethanol) is added to the resultant solution, a fentanyl compound, other additives (e.g., titanium oxide) and edible substances of (D) a water-soluble polyhydric alcohol, (B) a semi-synthetic water-soluble polymer compound, (A) a semi-synthetic water-insoluble polymer compound and (C) a synthetic water-soluble polymer compound are added and mixed while stirring to obtain a solution mixture. Subsequently, the solution mixture is spread on a release liner consisted of polyester film or the like and dried to obtain a film of 30 to 1,200 μm thick. This film is punched into pieces of a desired size to form film preparations (edible oral mucosal patch).

In the single-layer edible oral mucosal patch, the contents of individual ingredients (% by weight) areas follows. In a sustained-release edible oral mucosal patch which sustains for one hour or more in the oral cavity without disintegrating, the amount of (A) a semi-synthetic water-insoluble polymer compound is 3 to 20%, preferably 5 to 15%; the amount of (B) a semi-synthetic water-soluble polymer compound is 40 to 90%, preferably 50 to 80%; the amount of (C) a synthetic water-soluble polymer compound is 1 to 30%, preferably 5 to 20%; (D) a water-soluble polyhydric alcohol is 1 to 30%, preferably 5 to 20%; and (E) a pH-adjusting agent is 0.01 to 20%, preferably 0.1 to 17% of the total amount of the preparation. The amount of other additive optionally added is 0.1 to 10%, preferably 1 to 5% of the total amount of the preparation.

Furthermore, to reduce the disintegration time of the edible oral mucosal patch, the amount of (A) a semi-synthetic water-insoluble polymer compound is 1 to 10%, preferably 3 to 7%; the amount of (B) a semi-synthetic water-soluble polymer compound is 60 to 90%, preferably 70 to 90%, the amount of (C) a synthetic water-soluble polymer compound is 1 to 20%, preferably 5 to 15%; the amount of (D) a water-soluble polyhydric alcohol is 1 to 30%, preferably 5 to 20%; and the amount of (E) a pH-adjusting agent is 0.01 to 20%, preferably 0.1 to 17% of the total amount of the preparation. The amount of other additive optionally added is 0.1 to 10%, preferably 1 to 5% of the total amount of the preparation.

In the single-layer edible oral mucosal patch, it is preferable that an about 2% aqueous solution of the edible oral mucosal patch exhibits a pH of 4.0 to 8.0, in particular, 5.0 to 7.0. Therefore, the amount of a pH-adjusting agent is determined depending upon the pH-adjusting agent to be used and taking its characteristics into consideration.

### 2) Double-Layer (Non-Disintegrating Support Layer/Disintegrating Drug Layer) Preparation

To an appropriate amount of solvent (e.g., ethanol), edible substances of (D) a water-soluble polyhydric alcohol, (A) a semi-synthetic water-insoluble polymer compound, and (C) a synthetic water-soluble polymer compound, and other edible additive, if desired, are added and mixed while stirring to obtain a non-disintegrating support layer solution. Separately, to an appropriate amount of purified water, (E) a pH-adjusting agent is added and dissolved while stirring. To the resultant solution, an appropriate amount of solvent (e.g., ethanol) is added, and further a fentanyl compound and edible substances of (D) a water-soluble polyhydric alcohol, (B) a semi-synthetic water-soluble polymer compound and (C) a synthetic water-soluble polymer compound, and other additive, if necessary, are added and mixed while stirring to obtain a disintegrating drug layer solution.

Subsequently, the non-disintegrating support layer solution is spread on a release liner consisted of polyester film or the like and dried to obtain a film of about 10 to 200 μm thick. On this film, the disintegrating drug layer solution is spread and dried to obtain a film of 20 to 1,000 μm thick. As a result, a double-layer film of 30 to 1,200 μm thick can be obtained. The double-layer film is punched into pieces of a desired size to obtain film preparations.

The double-layer edible oral mucosal patch is applied to the oral mucosa such that the drug layer is in touch with the mucosa. Therefore, to distinguish the disintegrating drug layer clearly from the non-disintegrating support layer, for example, the support layer may be colored. To color the layer, a coloring agent, for example, red ferric oxide, may be added in an appropriate amount when the support layer solution is prepared. Furthermore, to distinguish two layers more clearly, for example, titanium oxide may be added in an appropriate amount when the disintegrating drug layer solution is prepared.

In the double-layer edible oral mucosal patch, the contents of individual ingredients (% by weight) are as follows.

The contents of individual ingredients of the non-disintegrating support layer are as follows. The amount of (A) a semi-synthetic water-insoluble polymer compound is 60 to 95%, preferably 70 to 95%; the amount of (C) a synthetic water-soluble polymer compound is 2 to 20%, preferably 5 to 15%; and (D) a water-soluble polyhydric alcohol is 2 to 20%, preferably 5 to 15%. The amount of other additive optionally added is 0.3 to 2%, preferably 0.5 to 1.5%.

The contents of individual ingredients of the disintegrating drug layer are as follows. The amount of main ingredient (a fentanyl compound) is 0.3 to 13%, preferably 0.5 to 10%; the amount of (B) a semi-synthetic water-soluble polymer compound is 45 to 90%, preferably 50 to 80%; the amount of (C) a synthetic water-soluble polymer compound is 3 to 15%, preferably 5 to 10%; the amount of (D) a water-soluble polyhydric alcohol is 2 to 20%, preferably 5 to 15%; and the amount of (E) a pH-adjusting agent is 0.01 to 20%, preferably 0.1 to 17%. The amount of other additive optionally added is 0.3 to 3.5%, preferably 1 to 3%.

The non-disintegrating support layer retains its shape at least for 5 hours after the drug layer is completely dissolved by the second fluid of the disintegration test method defined in the Japanese Pharmacopoeia. That is, after the drug layer is completely dissolved in the oral cavity, the support layer retains its shape without disintegrating. On the other hand, the disintegrating drug layer should be entirely dissolved in the oral cavity within the range from about 10 seconds to 24 hours, more preferably 10 seconds to 12 hours, which is determined using the above-described second fluid of the disintegration test method defined in the Japanese Pharmacopoeia.

In the double-layer edible oral mucosal patch, it is preferable that an about 2% aqueous solution of the edible oral mucosal patch, more specifically, an aqueous solution in which the disintegrating drug layer is dissolved or a dispersion solution (a suspension solution in which the support layer is suspended without dissolving), exhibits a pH of 4.0 to 8.0, in particular, 5.0 to 7.0. Therefore, the amount of a pH-adjusting agent is determined depending upon the pH-adjusting agent to be used and taking its characteristics into consideration.

### 3) Double-Layer or Triple-Layer (Disintegrating Support Layer/Disintegrating Drug Layer) Preparation

To an appropriate amount of purified water, (E) a pH-adjusting agent is added and dissolved while stirring. To the resultant solution, an appropriate amount of solvent (e.g., ethanol) is added, and further edible substances of (D) a water-soluble polyhydric alcohol, (A) a semi-synthetic water-insoluble polymer compound and (B) a semi-synthetic water-soluble polymer compound, and other edible additive, if necessary, are added and mixed while stirring to obtain a disintegrating support layer solution. Separately, to an appropriate amount of solvent (e.g., ethanol), a fentanyl compound and edible substances of (D) a water-soluble polyhydric alcohol, (B) a semi-synthetic water-soluble polymer compound and (C) a synthetic water-soluble polymer compound, and other additive, if necessary, are added and mixed while stirring to obtain a disintegrating drug layer solution.

Incidentally, to color the layer, a coloring agent, for example, red ferric oxide, may be added in an appropriate amount when the support layer solution is prepared and titanium oxide may be added in an appropriate amount when the drug layer solution is prepared similarly to the case of the double-layer preparation formed of a non-disintegrating support layer and a disintegrating drug layer.

In the edible oral mucosal patch formed of a disintegrating support layer and a disintegrating drug layer, the contents of individual ingredients (% by weight) are as follows.

The contents of individual ingredients of the disintegrating support layer are as follows. The amount of (A) a semi-synthetic water-insoluble polymer compound is 2 to 50%, preferably 5 to 30%; the amount of (B) a semi-synthetic water-soluble polymer compound is 30 to 95%, preferably 50 to 80%; and (D) a water-soluble polyhydric alcohol is 2 to 20%, preferably 5 to 15%. The amount of pH-adjusting agent is 0.01 to 20%, preferably 0.1 to 17%. The amount of other additive optionally added is 0.3 to 2%, preferably 0.5 to 1.5%.

The contents of individual ingredients of the disintegrating drug layer are as follows. The amount of main ingredient (a fentanyl compound) is 0.3 to 13%, preferably 0.5 to 10%; the amount of (B) a semi-synthetic water-soluble polymer compound is 45 to 90%, preferably 50 to 80%; the amount of (C) a synthetic water-soluble polymer compound is 3 to 15%, preferably 5 to 10%; the amount of (D) a water-soluble polyhydric alcohol is 2 to 20%, preferably 5 to 15%. The amount of other additive optionally added is 0.3 to 3.5%, preferably 1 to 3%.

In the edible oral mucosal patch formed of a disintegrating support layer and a disintegrating drug layer, it is preferable that an about 2% aqueous solution of the edible oral mucosal patch, more specifically, an aqueous solution in which the disintegrating drug layer is dissolved or a dispersion solution (a suspension solution in which the support layer is still suspended without dissolving), exhibits a pH of 4.0 to 8.0, in particular, 5.0 to 7.0, as is the same as the case of the double-layer preparation formed of a non-disintegrating support layer and a disintegrating drug layer. The amount of a pH-adjusting agent is determined depending upon the pH-adjusting agent to be used and taking its characteristics into consideration.

The disintegrating support layer and the disintegrating drug layer should be entirely dissolved within the range of 10 seconds to 24 hours, more preferably 10 seconds to 12 hours, which is determined using the second fluid of the disintegration test method defined in the Japanese Pharmacopoeia. The disintegrating support layer mentioned above should be disintegrated or dissolved within less than 5 hours after the drug layer is completely dissolved by the second fluid of the disintegration test method defined in the Japanese Pharmacopoeia. In other words, the support layer and the drug layer should be completely dissolved in the oral cavity. The dissolution-rate ratio of the disintegrating drug layer to the disintegrating support layer in the second fluid of the disintegration test method defined in the Japanese Pharmacopoeia is 5:1 to 1:5, preferably 3:1 to 1:3. This is because it is not only necessary for a pH-adjusting agent added to the support layer to control the pH in the oral cavity so as to absorb a drug easily, in order for the edible oral mucosal patch formed of the disintegrating support layer and the disintegrating drug layer to exhibit its performance more satisfactorily, but also necessary to release the drug by dissolving the drug layer before the drug-absorbable condition created by the pH-adjusting agent is destroyed by secretion of saliva.

The disintegrating support layer solution and the disintegrating drug layer solution are formed into a film preparation as follows.

The disintegrating support layer solution is spread on a release liner consisted of polyester film or the like and dried to obtain a film of about 5 to 200 μm thick. On this film, the disintegrating drug layer solution is spread and dried to obtain a film of 25 to 1,000 μm thick. As a result, a double-layer film of 30 to 1,200 μm thick can be obtained. The double-layer film is punched into pieces of a desired size to obtain film preparations.

Incidentally, it is preferable, than the above-mentioned double-layer structure, to use a triple-layer structure, which is formed by arranging the disintegrating support layers at both outer sides and the disintegrating drug layer between them. The disintegrating support layers placed at both outer sides can prevent the drug layer from adhering to a wrapping material during storage and to fingers when the drug layer is picked up by the fingers, thereby improving the handling of a patch by the user.

A triple-layer film preparation formed of disintegrating support layer/disintegrating drug layer/disintegrating support layer can be prepared in the following three methods.

A first method is performed only by coating. First, a disintegrating support layer solution is spread on a release liner consisted of polyester film or the like and dried to obtain a film of about 2.5 to 100 μm thick. On this film, a disintegrating drug layer solution is spread and dried to obtain a film of about 25 to 1000 μm thick (intermediate product 1) formed of the disintegrating support layer and the disintegrating drug layer. Further on the intermediate product 1, the disintegrating support layer solution is spread and dried to obtain a tripe-layer film of about 30 to 1,200 μm thick formed of support layer/drug layer/support layer. This film is punched into pieces of a desired size to obtain film preparations.

In a second method, two intermediate products 1 described above are prepared or the intermediate product 1 with the release liner of polyester or the like is cut in half in the longitudinal direction thereof to obtain two narrower half pieces. These two pieces are overlapped such that the drug layers of the two pieces of the intermediate product 1 are in contact with each other, passed through a pair of lamination rolls to laminate these two pieces at a preparation temperature of 30° C. to 95° C., preferably 50° C. to 70° C. (roll temperature of 30° C. to 150° C., preferably 30° C. to 80° C.) and a pressure of 0.05 to 1.5 MPa, preferably 0.1 to 0.7 Mpa to obtain a triple-layer film formed of support layer/drug layer/support layer. Thereafter, if necessary, the release liner is removed from either one of sides or both sides of the resultant laminated or multilayered film. The laminated film is then punched into pieces of a desired size to obtain film preparations.

In a third method, a disintegrating support layer solution is spread on a release liner consisted of polyester film or the like and dried to obtain a disintegrating support layer of about 2.5 to 100 μm thick (intermediate product 2). Separately, a disintegrating drug layer solution is spread on a release liner consisted of polyester film or the like and dried to obtain a disintegrating drug layer of about 12.5 to 500 μm thick (intermediate product 3).

These intermediate products 2 and 3 are overlapped such that the disintegrating support layer of intermediate 2 and the disintegration drug layer of intermediate 3 are in contact with each other, and passed through a pair of lamination rolls to laminate these intermediate products 2 and 3 at a preparation temperature of 30° C. to 95° C., preferably 50° C. to 70° C. (roll temperature of 30° C. to 150° C., preferably 30° C. to 80° C.) and a pressure of 0.05 to 1.5 MPa, preferably 0.1 to 0.7 MPa, to obtain a double-layer film of about 15 to 600 μm thick, formed of the support layer and the drug layer. Of the release liners attached on both sides, the release liner at the side of the drug layer is removed (intermediate product 4).

Two intermediate products 4 described above are prepared or the intermediate product 4 with the release liner is cut in half in the longitudinal direction thereof to obtain two narrower half pieces. These two pieces are overlapped such that the drug layers of the two pieces of the intermediate product 4 are in contact with each other, and passed through a pair of lamination rolls to laminate these two pieces at a preparation temperature of 30° C. to 95° C., preferably 50° C. to 70° C. (roll temperature of 30° C. to 150° C., preferably 30° C. to 80° C.) and a pressure of 0.05 to 1.5 MPa, preferably 0.1 to 0.7 MPa, to obtain a triple-layer film of 30 to 1,200 μm thick formed of support layer/drug layer/support layer. Thereafter, if necessary, the release liner is removed from either one of sides or both sides of the resultant laminated film. The resultant laminated or multilayered film is punched into pieces of a desired size to obtain film preparations.

In an edible oral mucosal patch obtained by laminating the support layer and the drug layer as described in the third method, the boundary between the support layer and the drug layer is more clearly distinguished than in the patch obtained by coating. Furthermore, since the support layer and the drug layer are not partially mixed with each other, a fentanyl compound is not influenced by (E) a pH-adjusting agent, thereby improving the stability of the fentanyl compound.

In addition, the lamination method such as the second and third methods contribute to improving quantitative accuracy required for a pharmaceutical preparation.

To explain more specifically, when a support layer solution and a drug layer solution (hereinafter generally referred to as preparation layer solutions) are spread and dried on a release liner (e.g. polyester film) in a plurality of times, the coating amount can be accurately controlled at a predetermined value at a first coating process by setting the clearance between a weir such as a doctor roll and the surface of the release liner at a predetermined dimension. However, the thickness of the dried first layer of the preparation formed in a drying process after the first coating process varies depending upon minor variation of the condition of the drying process and environmental conditions such as daily temperature and humidity. As a result, when a second coating is performed on the first layer, even if the clearance between the weir such as the doctor roll and the surface of the release liner is accurately set, the thickness of the second coating layer further varies depending upon the variation of the thickness of the first layer, since the coating thickness of the second layer solution is determined by the clearance between the upper surface of the dried first layer formed by the first coating process and the wire such as the doctor roll. Such an inaccurate coating amount of the preparation layer solution tends to increase as the number of spreading and drying processes increases. In addition, as the number of spreading and drying processes increases, the drying time increases. More specifically, the drying time for the second coating is about 1.5 times longer than that for the first coating. The drying time for the third coating is about twice longer than the first one.

According to a lamination method such as the second or third method, a desired number of preparation layers can be formed by laminating a plurality of extremely thin preparation layers instead of repeating processes of spreading and drying the preparation layer solutions on the release liner. In this way, a laminated or multilayered film-form edible oral mucosal patch can be prepared with good yield while overcoming the conventional problems. In addition, the quantitative accuracy required for a pharmaceutical preparation can be greatly improved.

In single-layer, double-layer and triple-layer edible oral mucosal patches, as the dimension and shape of a final film form preparation, a disk-shape of 15 mm or less in diameter, a square or a rectangle (those may have rounded corners) having a side of 15 mm or less, may be used. The dimension of 10 mm×12 mm is particularly preferable.

### EXAMPLES

First, Examples 1 and 2 of a single-layer edible oral mucosal patch will be explained below.

### Example 1

To an appropriate amount of purified water, 1.0 part by weight of sodium hydroxide was added and dissolved while stirring. To the solution, an appropriate amount of ethanol was added, and thereafter, 8.0 parts by weight of fentanyl citrate, 2.0 parts by weight of titanium oxide, 10.0 parts by weight of polyethylene glycol [macrogol 400 (Japanese Pharmacopoeia)], 40.0 parts by weight of hydroxypropyl cellulose (HPC), 20.0 parts by weight of ethyl cellulose (EC) and 19.0 parts by weight of polyvinyl pyrrolidone K90 (PVP K90) were added and mixed while stirring to obtain a drug solution. Subsequently, the drug solution was spread on a polyester release liner and dried to obtain a film of about 150 μm thick. The obtained film was punched into rectangular pieces of 10 mm×12 mm with rounded corners (radius r of the corner=2.8 mm) to obtain film preparations. The formulation of the film preparation is shown in Table 1.

Incidentally, in order to measure a pH value of the film preparations, 15 pieces of the rectangular film preparations of 10 mm×12 mm (total weight: 0.4 g) with rounded corners (r=2.8 mm) and 19.6 g of purified water were placed in a 50-mL centrifuging tube with screw cap. The centrifuging tube was tightly closed and shaken with a shaker at a rate of 200 times/min. Since the preparations were disintegrated to make a suspension solution 2.5 hours after the initiation of shaking, shaking was stopped and the pH of the suspended solution was measured. The pH value was 6.52.

### Example 2

A single-layer film preparation was obtained using the ingredients shown in Table 1 in the same manner as in Example 1. The pH was measured in the same manner as in Example 1, the pH value was 6.34.

TABLE 1
(% by weight)

| | | | | | Compara- | Compara- | Compara- | Compara- | Compara- |
|---|---|---|---|---|---|---|---|---|---|
| | | | | | tive | tive | tive | tive | tive |
| | Classi- | Ingredi- | Example | Example | Example | Example | Example | Example | Example |
| Layer | fication | ent | 1 | 2 | 1-A | 1-B | 1-C | 1-D | 1-E |
| Drug | | Fentanyl | 8.0 | 8.0 | 8.0 | 8.O | 8.0 | 8.O | 8.0 |
| layer | | citrate | | | | | | | |
| | | Titanium | 2.0 | 2.0 | 2.0 | 2.0 | 2.0 | 2.0 | 2.0 |
| | | oxide | | | | | | | |
| | D | Macrogol | 10.0 | 10.0 | 10.0 | 10.0 | 10.0 | — | 10.0 |
| | | 400 | | | | | | | |
| | B | HPC | 40.0 | 49.0 | 40.0 | — | 59.0 | 45.0 | 41.0 |
| | | MC | | 10.0 | | | | | |
| | A | EC | 20.0 | | — | 20.0 | 20.0 | 22.0 | 20.0 |
| | | HPMCP | | 5.0 | | | | | |
| | C | PVP K90 | 19.0 | 15.0 | 39.0 | 59.0 | — | 21.0 | 19.0 |
| | E | Sodium | 1.0 | 1.0 | 1.0 | 1.0 | 1.0 | 1.0 | — |
| | | hydroxide | | | | | | | |

Note)
HPC: hydroxypropyl cellulose
MC: methyl cellulose
EC: ethyl cellulose
HPMCP: hydroxypropylmethyl cellulose phthalate
PVP: polyvinyl pyrrolidone

### Comparative Examples 1-A, 1-B, 1-C, 1-D and 1-E

Single-layer film preparations according to Comparative Examples 1-A, 1-B, 1-C, 1-D and 1-E, which contain the same ingredients as in Example 1 (single-layer type edible oral mucosal patch) except that A, B, C, D and E ingredients were respectively eliminated from the formulation, were prepared based on the formulation shown in Table 1.

### Comparison Between Example 1 and Comparative Examples 1-A, 1-B, 1-C, 1-D and 1-E

In the edible oral mucosal patch according to the present invention containing a fentanyl compound as an active ingredient, there must be contained indispensably (A) a semi-synthetic water-insoluble polymer compound, (B) a semi-synthetic water-soluble polymer compound, (C) a synthetic water-soluble polymer compound, (D) a water-soluble polyhydric alcohol and (E) a pH-adjusting agent. A satisfactory preparation cannot be obtained if one of the five ingredients is not contained. To demonstrate this, the following tests: (a) wrapping material adhesion test, (b) punching test, (c) adhesion test, (d) crack test and (e) transmucosal permeability test were performed and the results are compared between Example 1 and Comparative Examples.

[Wrapping Material Adhesion Test (a)]

To examine the effect of the absence of the ingredient (A) semi-synthetic water-insoluble polymer compound on the performance of an edible oral mucosal patch, preparations of Example 1 and Comparative Example 1-A were subjected to the following wrapping material adhesion test.

### Test Method:

First, 10 pieces of rectangular preparations of 10 mm×12 mm with rounded corners (corner radius: r=2.8 mm) of each of Example 1 and Comparative Example 1-A were individually wrapped with a wrapping material formed of aluminum foil coated with polyacrylonitrile (PAN)-based sealant and stored at 60° C. for 7 days (equivalent to the storage at room temperature for one year). Then, the wrapping material was opened and picked up the preparation from the wrapping material wall by tweezers. The preparation that was picked up without deforming was recorded as “no adhesion to wrapping material.” The preparation deformed or broken, when picked up, was recorded as “adhesion to wrapping material.” The number of records “adhesion to wrapping material” was counted. The results are shown in Table 2.

TABLE 2
Results of wrapping material adhesion test (single-layer type)

| | | Comparative Example |
|---|---|---|
| | Example 1 | 1-A |
| Number of preparation adhered | 0 | 9 |
| to wrapping material | | |

As is apparent from Table 2, a preparation containing no (A) a semi-synthetic water-insoluble polymer compound adheres to a wrapping material with a probability of 90%, and thus difficult to be taken out.

[Punching Test (b)]

The performance of an edible oral mucosal patch may be affected if (B) a semi-synthetic water-soluble polymer compound is not contained. To examine the effect of ingredient (B), preparations of Example 1 and Comparative Example 1-B were subjected to the following punching test.

### Test Method:

A film preparation of about 150 μm thick obtained by coating a drug solution on a polyester release liner was punched out by a die of a punching machine to obtain rectangular preparations of 10 mm×12 mm with rounded corners (corner radius: r=2.8 mm). There were preparations in which a crack and/or fracture was formed by the impact when punched out. The number of preparations having a crack and/or fracture was counted. The test was repeated 10 times. The results are shown in Table 3.

TABLE 3
Number of preparations with punching fracture (single-layer type)

| | | Comparative Example |
|---|---|---|
| | Example 1 | 1-B |
| Number of punching | 0 | 10 |
| failure | | |

From the results of Table 3, it is found that a preparation containing no (B) a semi-synthetic water-soluble polymer compound becomes fragile. As a result, punching failure, such as occurrence of crack or fracture, of a preparation was recognized in a punching process.

[Adhesion Test (c)]

The performance of an edible oral mucosal patch may be affected if (C) a synthetic water-soluble polymer compound is not contained. To examine the effect of the ingredient (C), preparations of Example 1 and Comparative Example 1-C were subjected to the following adhesion test.

### Test Method:

(1) Measuring Apparatus

As an adhesion measuring apparatus, a rheometer (RT-3020D-CW type, manufactured by Rheotech Co., Ltd.) shown in FIG. 1 was used. FIG. 1 is a schematic perspective view of the measuring apparatus. A rod 2 is provided at the center of the upper surface of a horizontal disk 1 (30 mm diameter) formed of a phenol resin. The upper end of the rod 2 is connected to a fixed load cell 3. On the other hand, a holding table 4 is configured to move up and down constantly at a desired rate.

At the lower surface of the disk 1, a membrane filter 5 (Type A500A090C, pore diameter: 5.0 μm, manufactured by Advantec Toyo Kaisha, Ltd.) cut so as to have the same size and shape as those of the disk 1 is fixed by means of a double faced adhesive tape. The membrane filter simulates human mucosa.

(2) Measurement Method

A single preparation 6 of 10 mm×12 mm with rounded corners (corner radius: r=2.8 mm) was adhered onto the center of the holding table 4 by means of a double faced adhesive tape. After 0.1 mL of water was added dropwise on the center portion of the preparation 6, the holding table 4 was moved up and pressed against the membrane filter 5 on the lower surface of the disk 1 under a load of 2 kg for 20 seconds. Immediately after that, the holding table 4 was moved down at a rate of 5 mm/min to peel off the preparation 6 from the membrane filter 5. The weight of the load cell at that time was measured. The test was repeated three times. The arithmetic average (arithmetic mean) was regarded as a measurement value. The results are shown in Table 4.

TABLE 4
Results of adhesion test (single-layer type)

| | | Comparative Example |
|---|---|---|
| | Example 1 | 1-C |
| Average value | 1.19 kg | 0.49 kg |

From the results of Table 4, it is found that a preparation containing no (C) a synthetic water-soluble polymer compound decreases in adhesive strength by about 60%. As a result, the preparation has insufficient adhesive strength.

[Crack Test (d)]

The performance of an edible oral mucosal patch may be affected if (D) a water-soluble polyhydric alcohol is not contained. To examine the effect of the ingredient (D), preparations of Example 1 and Comparative Example 1-D were subjected to the following crack test.

### Test Method:

A film preparation before punching out into pieces (10 mm×12 mm (r=2.8 mm)) was cut into pieces of 2 cm×10 cm. The piece was placed on a table and folded while one side of the piece was fixed. The folding angle at which the piece is broken was measured. The results are shown in FIG. 5.

TABLE 5
Crack test (single-layer type)

| | | Comparative Example |
|---|---|---|
| | Example 1 | 1-D |
| Crack test | Not crack when folded | 80° |
| | into 180° | |

From the results of Table 5, it is found that a preparation containing no (D) a water-soluble polyhydric alcohol becomes fragile and cracks when it is folded into 80°.

### [Transmucosal Permeability Test (e), Hamster Cheek Pouch in Vitro Test]

The performance of an edible oral mucosal patch may be affected if (E) a pH-adjusting agent is not contained. To examine the effect of the ingredient (E), preparations of Example 1 and Comparative Example 1-E were subjected to the following transmucosal permeability test.

### Test Method:

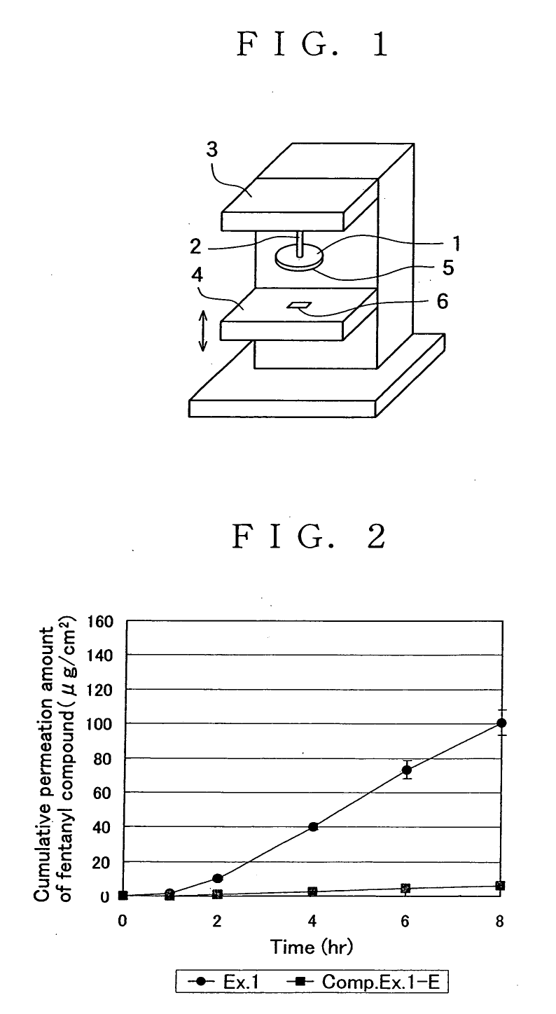

An aqueous solution of urethane was injected to the abdominal cavity of a hamster to anesthetize the hamster. Thereafter, the cheek pouch of the hamster was taken out from the mouth so as to turn inside out. After washing with physiological saline, the cheek pouch was fixed on a diffusion cell (application area: 0.95 cm², cell volume: 2.5 mL) such that its keratinocyte surface faced the donor side. 2.5 mL of isotonic phosphate buffer (pH7.4) was placed at the acceptor side, and each of test substances (preparations of Example 1, Comparative Example 1-E) was attached to the donor side. Warm water of 37° C. was circulated through the jacket of the cell to keep the temperature of internal liquid was maintained constant. Every after 1, 2, 4, 6 and 8 hours, an aliquot of 0.5 ml was taken from the internal liquid and subjected to high performance liquid chromatography to quantitatively determine the amount of a fentanyl compound in the samples taken above. The cumulative amount of a fentanyl compound permeated through the cheek pouch mucosa was calculated. The results are shown in Table 6 and FIG. 2.

TABLE 6
Cumulative permeation amount of fentanyl compound (μg/cm²)

| Formula- | Time (hr) | | | | | | |
|---|---|---|---|---|---|---|---|
| tion No. | 0 | 1 | 2 | 4 | 6 | 8 | pH |
| Example 1 | 0 | 1.68 | 10.05 | 40.22 | 73.27 | 100.79 | 6.52 |
| Comparative | 0 | 0.20 | 0.77 | 2.51 | 4.34 | 6.10 | 4.13 |
| Example 1-E | | | | | | | |

As is apparent from the results of Table 6 and FIG. 2, the edible oral mucosal patch of the present invention is particularly excellent in transmucosal permeability of a fentanyl compound compared that of Comparative Example. It is therefore found that the preparation containing no (E) a pH-adjusting agent significantly decreases in transmucosal permeability of a fentanyl compound.

Now, Examples 3 to 10 with respect to an edible oral mucosal patch having a non-disintegrating support layer containing (A) a semi-synthetic water-insoluble polymer compound, (C) a synthetic water-soluble polymer compound and (D) a water-soluble polyhydric alcohol; and a disintegrating drug layer containing a fentanyl compound, (B) a semi-synthetic water-soluble polymer compound, (C) a synthetic water-soluble polymer compound, (D) a water-soluble polyhydric alcohol and (E) a pH-adjusting agent, will be explained.

### Example 3

To an appropriate amount of ethanol, 10.0 parts by weight of polyethylene glycol [macrogol 400 (Japanese Pharmacopeia)], 1.0 parts by weight of red ferric oxide, 80.0 parts by weight of ethyl cellulose (EC) and 9.0 parts by weight of polyvinyl pyrrolidone K90 (PVP K90) were added and mixed while stirring to obtain a non-disintegrating support layer solution. Separately, to an appropriate amount of purified water, 1.0 part by weight of sodium hydroxide was added and dissolved while stirring. To the solution, an appropriate amount of ethanol was added, and thereafter, 8.0 parts by weight of fentanyl citrate, 2.0 parts by weight of titanium oxide, 10.0 parts by weight of macrogol 400, 71.5 parts by weight of hydroxypropyl cellulose (HPC) and 7.5 parts by weight of polyvinyl pyrrolidone K90 (PVP K90) were added and mixed while stirring to obtain a disintegrating drug layer solution. Subsequently, the non-disintegrating support layer solution was spread on a polyester release liner and dried to obtain a film of 25 μm thick. On the film, the disintegrating drug layer solution was spread and dried to obtain a film of about 130 μm thick. The thickness of the resultant double-layer film was about 155 μm. The resultant film was punched into rectangular pieces (10 mm×12 mm) with rounded corners (corner radius: r=2.8 mm) to obtain film preparations. The formulation of the film preparation is shown in Table 7.

Incidentally, to measure a pH value of the film preparation, 15 pieces of the rectangular film preparations of 10 mm×12 mm (total weight: 0.4 g) with rounded corners (r=2.8 mm) and 19.6 g of purified water were placed in 50 mL of a centrifuging tube with screw cap. The centrifuging tube was tightly closed and shaken by a shaker at a rate of 200 times/min. Since the drug layers were completely dissolved 2.5 hours after the initiation of shaking, shaking was stopped. The support layers were still undissolved. The pH of the solution in which undissolved support layers were suspended was measured to obtain a pH value of 6.22.

Furthermore, to confirm that the non-disintegrating support layer maintains its shape without disintegrating at least for 5 hours after the drug layer is completely dissolved with the second fluid of the disintegration test method defined in the Japanese Pharmacopoeia, the following test was performed.

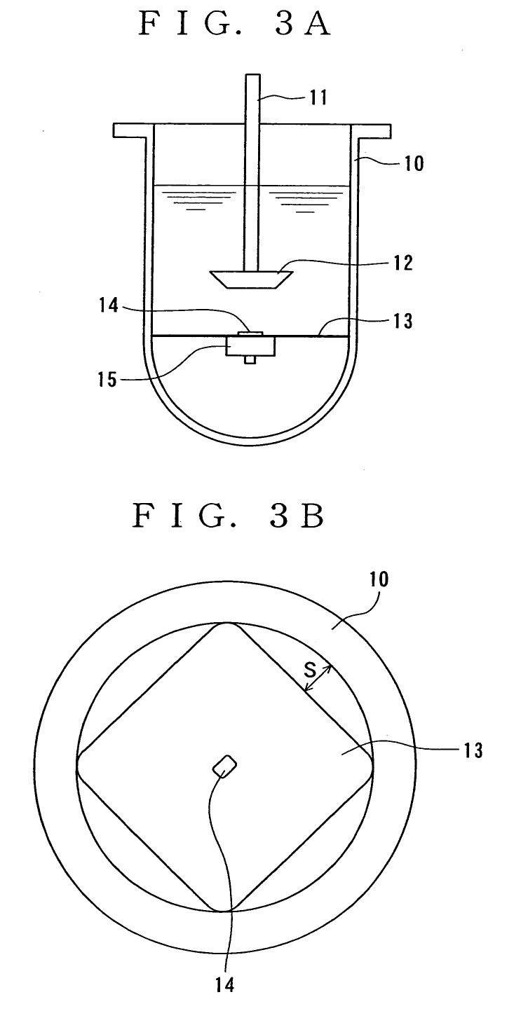

A test solution was prepared by adding 118 mL of 0.2 mol/L sodium hydroxide solution and water to 250 ml of 0.2 mol/L potassium dihydrogenphosphate solution to bring a final volume of 1000 mL. Using 500 mL of the test solution, i.e. the second liquid, a test was performed at a temperature of 37±0.5° C. at a paddle rotation number of 50 per minute in accordance with the U.S. Pharmacopeia <724> DRUG RELEASE Transdermal Delivery Systems-General Drug Release Standards (corresponding to the Japanese Pharmacopeia, the dissolution test method, Method 2 (Paddle method)). The apparatus used in the test is shown in FIGS. 3A and 3B. This apparatus has a container 10 having a semispherical bottom and a paddle 12 attached to the lower end of a rotation shaft 11 rotated by a motor (not shown). The container 10 contains the aforementioned test solution. A celluloid plate 13 of 80 mm×80 mm is arranged between the lower end of the paddle 12 and the bottom of the container 10. On the center portion of the upper surface of the celluloid plate 13, a corner-rounded rectangular film preparation 14 (10 mm×12 mm, r=2.8 mm) is adhered by a double faced adhesive tape with the support layer upward (outer side). On the other hand, on the center portion of the lower surface of the celluloid board, a weight 15 is allowed to adhere by a synthetic polymer adhesive agent. In this container, as shown in FIG. 3B, the maximum distance S between a side of the celluloid plate 13 and the inner peripheral surface of the container 10 is set at 10 mm.

The test was performed as follow. While the test solution of the container 10 is stirred by rotating the paddle 12, a degree of the dissolution of the film preparation 14 on the celluloid plate 13 was visually observed. About a half of the drug layer remained after 8 hours from the initiation of stirring, so that visual observation was stopped and stirring was continued. Next morning (23 hours after the initiation of stirring), the drug layer was dissolved and disappeared, whereas the support layer floated in the test solution without disintegrating. The paddle was rotated for further 5 hours. However, no appearance change of the support layer was observed. From this, it was demonstrated that the support layer remains at least 5 hours without disintegrating after the drug layer is completely dissolved with the second fluid of the disintegration test method defined in the Japanese Pharmacopeia.

### Examples 4 to 10

A film preparation was obtained using the ingredients shown in Table 7 in the same manner as in Example 3.

TABLE 7
(% by weight)

| | Classi- | | Example | Example | Example | Example | Example | Example | Example | Example |
|---|---|---|---|---|---|---|---|---|---|---|
| Layer | fication | Ingredient | 3 | 4 | 5 | 6 | 7 | 8 | 9 | 10 |
| Support | D | Macrogol 400 | 10.0 | 10.0 | | 10.0 | 10.0 | | | |
| layer | | Macrogol | | | 10.0 | | | | | |
| | | 4000 | | | | | | | | |
| | | PG | | | | | | 10.0 | | |
| | | Macrogol | | | | | | | | 10.0 |
| | | 6000 | | | | | | | | |
| | | D-sorbitol | | | | | | | 10.0 | |
| | | Red ferric | 1.0 | 1.0 | 1.0 | 1.0 | 1.0 | 1.0 | 1.0 | 1.0 |
| | | oxide | | | | | | | | |
| | A | EC | 80.0 | 80.0 | 80.0 | | 80.0 | 80.0 | | 80.0 |
| | | HPMCP | | | | 80.0 | | | 80.0 | |
| | C | PVP K90 | 9.0 | 9.0 | 9.0 | 9.0 | 9.0 | | 9.0 | |
| | | PVA | | | | | | 9.0 | | 9.0 |
| Drug | | Fentanyl | 8.0 | 8.0 | 8.0 | 8.0 | 8.0 | 8.0 | 4.0 | 0.8 |
| layer | | citrate | | | | | | | | |
| | | Titanium | 2.0 | 2.0 | 2.0 | 2.0 | 2.0 | 2.0 | 2.0 | 2.0 |
| | | oxide | | | | | | | | |
| | D | Macrogol 400 | 10.0 | 10.0 | 10.0 | 10.0 | | 10.0 | | 10.0 |
| | | PG | | | | | 10.0 | | | |
| | | D-sorbitol | | | | | | | 10.0 | |
| | B | HPC | 71.5 | | 57.5 | 62.5 | | 57.5 | 76.3 | 79.5 |
| | | MC | | | | | 71.5 | | | |
| | | HPMC | | 72.3 | | | | | | |
| | C | PVP K90 | 7.5 | 7.5 | | 7.5 | 7.5 | | 7.5 | 7.5 |
| | | CVP | | | 7.5 | | | 7.5 | | |
| | B(C) | PAA-Na | | | | | 1.0 | | | |
| | E | Sodium | 1.0 | 0.2 | | | | 15.0 | 0.2 | 0.2 |
| | | hydroxide | | | | | | | | |
| | | Sodium | | | | 10.0 | | | | |
| | | bicarbonate | | | | | | | | |
| | | Sodium | | | 15.0 | | | | | |
| | | acetate | | | | | | | | |

Note)
PG: propylene glycol
EC: ethyl cellulose
HPMCP: hydroxypropylmethyl cellulose phthalate
PVP: polyvinyl pyrrolidone
PVA: polyvinyl alcohol
HPC: hydroxypropyl cellulose
MC: methyl cellulose
HPMC: hydroxypropylmethyl cellulose
CVP: carboxyvinyl polymer
PAA-Na: sodium polyacrylate

### Comparative Examples 3-1A, 3-1C, 3-1D, 3-2B, 3-2C, 3-2D and 3-2E

Film preparations according to Comparative Examples 3-1A, 3-1C, 3-1D, 3-2B, 3-2C, 3-2D and 3-2E, each of which an indispensable ingredient of the support layer and an indispensable ingredient of the drug layer of Example 3 were eliminated, were prepared in accordance with the formulation shown in Table 8.

TABLE 8
(% by weight)

| | | | | Compara- | Compara- | Compara- | Compara- | Compara- | Compara- | Compara- |
|---|---|---|---|---|---|---|---|---|---|---|
| | | | | tive | tive | tive | tive | tive | tive | tive |
| | Classi- | | Example | Example | Example | Example | Example | Example | Example | Example |
| Layer | fication | Ingredient | 3 | 3-1A | 3-1C | 3-1D | 3-2B | 3-2C | 3-2D | 3-2E |
| Support | D | Macrogol 400 | 10.0 | 10.0 | 10.0 | — | 10.0 | 10.0 | 10.0 | 10.0 |
| layer | | Red ferric | 1.0 | 1.0 | 1.0 | 1.0 | 1.0 | 1.0 | 1.0 | 1.0 |
| | | oxide | | | | | | | | |
| | A | EC | 80.0 | — | 89.0 | 89.1 | 80.0 | 80.0 | 80.0 | 80.0 |
| | B | HPC | — | 80.0 | — | — | — | — | — | — |
| | C | PVP K90 | 9.0 | 9.0 | — | 9.9 | 9.0 | 9.0 | 9.0 | 9.0 |
| Drug | | Fentanyl | 8.0 | 8.0 | 8.0 | 8.0 | 8.0 | 8.0 | 8.0 | 8.0 |
| layer | | citrate | | | | | | | | |
| | | Titanium | 2.0 | 2.0 | 2.0 | 2.0 | 2.0 | 2.0 | 2.0 | 2.0 |
| | | oxide | | | | | | | | |
| | D | Macrogol 400 | 10.0 | 10.0 | 10.0 | 10.0 | 10.0 | 10.0 | — | 10.0 |
| | B | HPC | 71.5 | 71.5 | 71.5 | 71.5 | — | 79.0 | 81.5 | 72.5 |
| | C | PVP K90 | 7.5 | 7.5 | 7.5 | 7.5 | 79.0 | — | 7.5 | 7.5 |
| | E | Sodium | 1.0 | 1.0 | 1.0 | 1.0 | 1.0 | 1.0 | 1.0 | — |
| | | hydroxide | | | | | | | | |

### Comparison Between Example 3 and Comparative Examples 3-1A, 3-1C, 3-1D, 3-2B, 3-2C, 3-2D and 3-2E

In the edible oral mucosal patch according to Example 3 comprising a non-disintegrating support layer containing (A) a semi-synthetic water-insoluble polymer compound, (C) a synthetic water-soluble polymer compound and (D) a water-soluble polyhydric alcohol; and a disintegrating drug layer containing a fentanyl compound, (B) a semi-synthetic water-soluble polymer compound, (C) a synthetic water-soluble polymer compound, (D) a water-soluble polyhydric alcohol and (E) a pH-adjusting agent, the ingredients (A), (C) and (D) in the non-disintegrating support layer and the ingredients (B), (C), (D) and (E) in the disintegrating drug layer are indispensable. A satisfactory preparation cannot be obtained if one of these ingredients is not contained. To demonstrate this, the following tests: (f) dissolution test, (c) adhesion test, (g) delamination test between a support layer and a drug layer, (h) crack formation test, and (e) transmucosal permeability test were performed and the results of Example and Comparative Examples were compared. The tests represented by the same reference symbols mean that the tests were performed in the same manner.

[Dissolution Test (f)]

The performance of an edible oral mucosal patch may be affected if the non-disintegrating support layer contains no (A) a semi-synthetic water-insoluble polymer compound and the disintegrating drug layer contains no (B) a semi-synthetic water-soluble polymer compound. To examine the effects of the ingredients upon the patch, the preparations of Example 3 and Comparative Examples 3-1A and 3-2B were subjected to the following dissolution test.

### Test Method:

As a test solution, 500 mL of purified water was used. A test was performed at a temperature of 37±0.5° C. at a paddle rotation number of 50 per minute in accordance with the U.S. Pharmacopeia <724> DRUG RELEASE Transdermal Delivery Systems-General Drug Release Standards (corresponding to the Japanese Pharmacopeia, the dissolution test method, Method 2 (paddle method)). The apparatus used in the test is the same as that shown in FIGS. 3A to 3B. On the upper surface of the celluloid plate 13 of 80 mm×80 mm, which has the weight 15 adhered on the lower surface with a synthetic polymer adhesive agent, a test patch was adhered by a double faced adhesive tape with the support layer upward (outer side). In this way, the test patch was placed between the lower end of the paddle 12 and the bottom of the container 10.

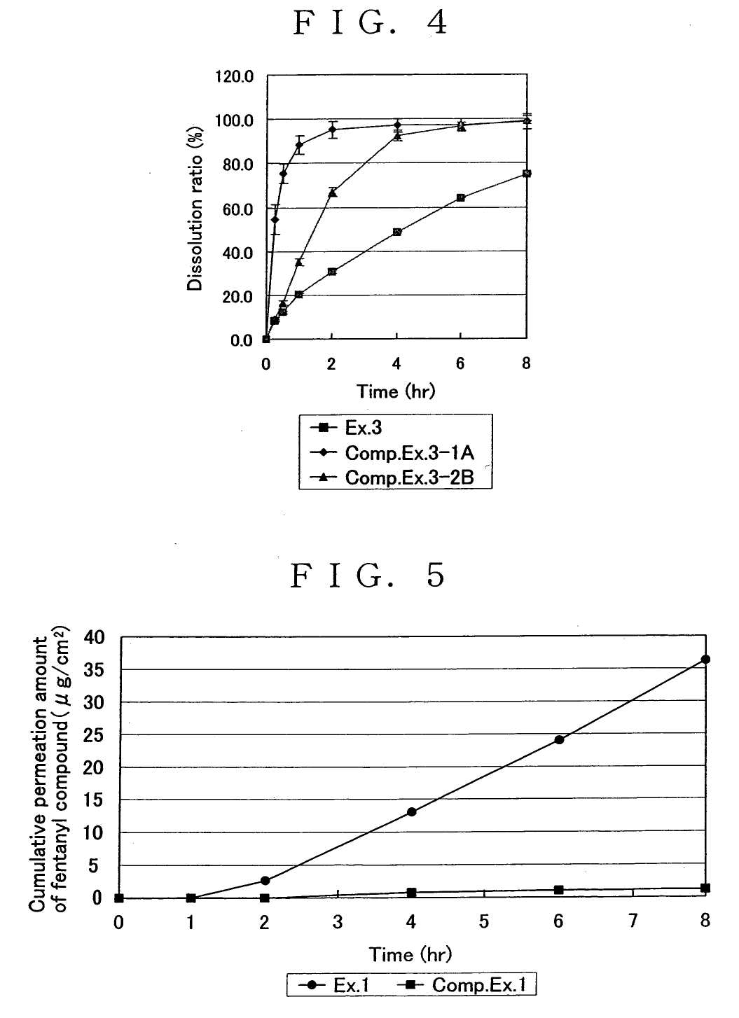

The test was performed while stirring the test solution in the container by rotating the paddle. Every after 15 and 30 minutes, one hour, 2, 4, 8 and 24 hours, an aliquot of 2 mL was taken from the test solution. Immediately after the aliquot was taken, 2 mL of fresh test solution warmed up to 37±0.5° C. was added to the container. The test solution thus taken was filtrated by a dissolution-test filter and subjected to high performance liquid chromatography to measure the amount of a fentanyl compound dissolved each time. The dissolution ratio was calculated and the results are shown in the graph of FIG. 4.

From FIG. 4, the patch of Example 3 shows a dissolution ratio of about 70% even 8 hours after the initiation of the test. Therefore, the patch of Example 3 is excellent in sustain-release compared to the patch of Comparative Example 3-1A. In other words, the results exhibit that sustained release of a drug can be attained by adding (A) a semi-synthetic water-insoluble polymer compound to the non-disintegrating support layer and the drug-release sustaining time can be controlled. In the patch of Comparative Example 3-1A containing no (A) a semi-synthetic water-insoluble polymer compound in the support layer, the support layer was disintegrated earlier than the drug layer, with the result that the dissolution of the drug layer cannot be suppressed. For the reason that sustained drug release cannot be attained, the patch of Comparative Example 3-1A is not preferable.

Comparative Example 3-2B containing no (B) a semi-synthetic water-soluble polymer compound in the disintegrating drug layer is not preferable since shape of the preparation was not maintained, allowing main ingredient to dissolve in an early stage.

[Adhesion Test (c)]

The performance of an edible oral mucosal patch may be affected if (C) a synthetic water-soluble polymer compound is not contained in a disintegrating drug layer. To examine the effect of the ingredient (C) on the patch, preparations of Example 3 and Comparative Example 3-2C were subjected to the following adhesion test.

### Test Method:

Using the measurement apparatus shown in FIG. 1, a preparation was adhered by a double faced adhesive tape onto the center portion of the holding table with the drug layer faced upward. The adhesive strength of the preparation was measured in the same procedure as described above. The results are shown in Table 9.

TABLE 9
Results of adhesion test (double-layer type)

| | | Comparative Example |
|---|---|---|
| | Example 3 | 3-2C |
| Average value | 0.98 kg | 0.81 kg |

From the results of Table 9, it is found that a preparation containing no (C) a synthetic water-soluble polymer compound in the disintegrating drug layer decreases in adhesive strength of the drug layer by about 20%.

[Delamination Test Between Support Layer and Drug Layer (g)]

The performance of an edible oral mucosal patch may be affected if (C) a synthetic water-soluble polymer compound is not contained in a non-disintegrating support layer and (D) a water-soluble polyhydric alcohol is not contained in a disintegrating drug layer. To examine the effect of the ingredients (C) and (D) on the patch, preparations of Example 3, Comparative Examples 3-1C and 3-2D were subjected to the following delamination test.

### Test Method:

A non-disintegrating support layer solution was spread on a polyester release liner and dried to obtain a non-disintegrating support layer of 25 μm thick. On this layer, a disintegrating drug layer solution was spread and dried to obtain a disintegrating drug layer of about 130 μm. The double layer preparation film was about 155 μm thick. The preparation film was punched out by a die of a punching machine to obtain rectangular patches of 10 mm×12 mm with rounded corners (corner radius: r=2.8 mm). If delamination between the support layer and the drug layer occurred due to the impact of punching out in not less than half of the patch area, the patch was regarded as a “delaminated patch.” The number of delaminated patches were counted. The test was repeated 10 times. The results are shown in Table 10.

TABLE 10
Number of preparations having delamination between
support layer and drug layer in not less than
half of patch area (double-layer type)

| | | Comparative | Comparative |
|---|---|---|---|
| | Example 3 | Example 3-1C | Example 3-2D |
| Number of delamination | 0 | 6 | 10 |
| between support layer and | | | |
| drug layer | | | |

From the results of Table 10, it is found that delamination takes place between the support layer and the drug layer with a high probability if (C) a synthetic water-soluble polymer compound is not contained in anon-disintegrating support layer or (D) a water-soluble polyhydric alcohol is not contained in a disintegrating drug layer.

[Crack Formation Test (h)]

The performance of an edible oral mucosal patch may be affected if (D) a water-soluble polyhydric alcohol is not contained in a non-disintegrating support layer. To examine the effect of the ingredient (D) on the patch, preparations of Example 3 and Comparative Example 3-1D were subjected to the following crack formation test.

### Test Method:

Ten pieces of preparations punched into a size of 10 mm×12 mm (r=2.8 mm) were stored at 60° C. for 2 days (equivalent to the storage at room temperature for several months). A piece of the preparations was folded in half such that both ends of its longitudinal side were in contact with each other and immediately opened flat. The number of cracks formed in the surface of the support layer was counted. The test was repeated ten times. The results are shown in Table 11.

TABLE 11
Crack formation rate (double-layer type)

| | | Comparative Example |
|---|---|---|
| | Example 3 | 3-1D |
| Number of preparations having | 0 | 10 |
| a crack when folded in half | | |

From the results of Table 11, it is found that crack is formed in the support layer with a high probability by folding a preparation due to impact if (D) a water-soluble polyhydric alcohol is not contained in the non-disintegrating support layer.

### [Transmucosal Permeability Test (e): Hamster Cheek Pouch in Vitro Test]

The performance of an edible oral mucosal patch may be affected if (E) a pH-adjusting agent is not contained in a disintegrating drug layer. To examine the effect of the ingredient (E) on the patch, preparations of Example 3 and Comparative Example 3-2E were subjected to the following transmucosal permeability test.

### Test Method:

The cumulative permeation amount of a fentanyl compound passed through the cheek pouch mucosa was calculated in the same manner as the transmucosal permeability test (e) using a hamster cheek pouch described above. The results are shown in Table 12 and FIG. 5.

TABLE 12
Cumulative permeation amount of fentanyl compound (μg/cm²)

| Formula- | Time (hr) | | | | | | |
|---|---|---|---|---|---|---|---|
| tion No. | 0 | 1 | 2 | 4 | 6 | 8 | pH |
| Example 3 | 0 | 0 | 2.63 | 13.53 | 24.43 | 35.57 | 5.60 |
| Comparative | 0 | 0.065 | 0.13 | 0.50 | 0.815 | 1.13 | 3.76 |
| Example 3-2E | | | | | | | |

From the results of Table 12 and FIG. 5, it is found that the edible oral mucosal patch of the present invention is remarkably excellent in transmucosal permeability of a fentanyl compound compared that of Comparative Example.

### Summary of Various Test Results in Examples 1 to 10 and Comparative Examples

From the various test results in which the preparations according to Examples 1 to 10 of the present invention are compared to those of Comparative Examples each of which one of the indispensable ingredients (A), (B), (C), (D) and (E) were eliminated, problems taking place in the preparations of Comparative Examples are summarized in Table 13. It is found that if even one of the five indispensable ingredients is not contained, the problems listed in Table 13 takes place, with the result that a satisfactory preparation cannot be obtained. In other words, it can be said that the oral mucosal patches of Examples 1 to 10 have overcome all of these problems. Note that reference symbols (a) to (h) in Table 13 correspond to those used in various tests described above.

TABLE 13

| Ingredient not | Single-layer | Double-layer type | |
|---|---|---|---|
| contained | type | Support layer | Drug layer |
| (A) a semi-synthetic | (a) | (f) | |
| water-insoluble | Adhesion to | Early | |
| polymer compound | wrapping | dissolving | |
| | material | | |
| (B) a semi-synthetic | (b) | | (f) |
| water-soluble | Crack by | | Early |
| polymer compound | punching | | dissolving |
| (C) a synthetic | (c) | (g) | (c) |
| water-soluble | Poor adhesion | Delami- | Poor adhesion |
| polymer compound | About 60% | nation | About 20% |
| | decrease | | decrease |
| (D) a water-soluble | (d) | (h) | (g) |
| polyhydric alcohol | Crack and/or | Crack | Delamination |
| | fracture | formation | |
| (E) a pH-adjusting | (e) | | (e) |
| agent | Poor | | Poor |
| | transmucosal | | transmucosal |
| | permeability | | permeability |

Next, Examples 11 and 12 with respect to edible oral mucosal patches formed of a disintegrating support layer comprising (A) a semi-synthetic water-insoluble polymer compound, (B) a semi-synthetic water-soluble polymer compound and (D) a water-soluble polyhydric alcohol; and a disintegrating drug layer comprising a fentanyl compound, (B) a semi-synthetic water-soluble polymer compound, (C) a synthetic water-soluble polymer compound and (D) a water-soluble polyhydric alcohol, will be explained.

### Example 11

To an appropriate amount of purified water, 1.2 parts by weight of sodium hydroxide was added and dissolved while stirring. To the solution, an appropriate amount of ethanol was added, and thereafter, 10.0 parts by weight of polyethylene glycol [macrogol 400 (Japanese Pharmacopoeia)], 2.0 parts by weight of titanium oxide, 26.0 parts by weight of hydroxypropylmethyl cellulose phthalate (HPMCP) and 60.8 parts by weight of hydroxypropyl cellulose (HPC) were added and mixed while stirring to obtain a disintegrating support layer solution. Separately, to an appropriate amount of ethanol, 1.98 parts by weight of fentanyl citrate, 2.0 parts by weight of titanium oxide, 10.0 parts by weight of macrogol 400, 78.53 parts by weight of hydroxypropyl cellulose (HPC) and 7.5 parts by weight of polyvinyl pyrrolidone K90 (PVP K90) were added and mixed while stirring to obtain a disintegrating drug layer solution.

Subsequently, the disintegrating support layer solution was spread on a polyester release liner and dried to obtain a film of 5 μm thick. On the resultant film, the disintegrating drug layer solution was spread and dried to obtain a film of 45 μm thick. The thickness of the resultant double-layer film was 50 μm (intermediate product a). The intermediate product a with the polyester release liner was cut in half along its longitudinal direction to obtain two narrower pieces. These two pieces were overlapped such that the drug layers of the two intermediate products a were in contact with each other, passed through a pair of lamination rolls to laminate these two pieces at a preparation temperature of 60° C., and a pressure of 0.3 MPa to obtain a triple-layer film (100 μm thick) formed of support layer/drug layer/support layer. Thereafter, if necessary, the release liner was removed from either one or both sides of the resultant laminated or multilayered film. The resultant film was punched into rectangular pieces of 10 mm×12 mm with rounded corners (corner radius: r=2.8 mm) to obtain film preparations. The formulation of the film preparation is shown in Table 14.

TABLE 14
(% by weight)

| | Classi- | | | | Comparative |
|---|---|---|---|---|---|
| Layer | fication | Ingredient | Example 11 | Example 12 | Example 11 |
| Support | D | Macrogol 400 | 10.0 | 10.0 | 10.0 |
| layer | | Titanium oxide | 2.0 | 2.0 | 2.0 |
| | A | HPMCP | 26.0 | 27.2 | 27.2 |
| | B | HPC | 60.8 | 52.3 | 60.8 |
| | E | Sodium hydroxide | 1.2 | — | — |
| | | Trisodium phosphate dodecahydrate | — | 8.5 | — |
| Drug | | Fentanyl citrate | 1.98 | 1.98 | 1.98 |
| layer | | Titanium oxide | 2.0 | 2.0 | 2.0 |
| | D | Macrogol 400 | 10.0 | 10.0 | 10.0 |
| | B | HPC | 78.53 | 78.53 | 78.23 |
| | C | PVP K90 | 7.5 | 7.5 | 7.5 |
| | E | Sodium hydroxide | — | — | 0.3 |

Incidentally, to measure the pH value of the film preparation, 25 pieces of rectangular film preparations of 10 mm×12 mm (total weight: 0.4 g) with rounded corners (r=2.8 mm) and 19.6 g of purified water were placed in 50 mL of a centrifuging tube with screw cap. The centrifuging tube was tightly closed and shaken by a shaker at a rate of 200 times/min. Since the drug layers were completely dissolved and the support layers were disintegrated to make a suspension solution 30 minutes after the initiation of shaking, shaking was stopped. When the pH of the suspended solution was measured, it was 5.48.

To confirm the dissolution or disintegration states of the drug layer and the support layer with the second fluid of the disintegration test method defined in the Japanese Pharmacopoeia, the same test was performed in the same manner as in Example 3 by using the apparatus shown in FIGS. 3A and 3B. As a result, both the drug layer and the support layer were completely dissolved after one hour from the initiation of stirring by the paddle.

Furthermore, to obtain the dissolution-rate ratio of the support layer and the drug layer of Example 11, a film preparation punched into a rectangular shape of 10 mm×12 mm with rounded corners (r=2.8 mm) was prepared in accordance with the same formulation as in Example 11 and shown in Table 15 such that the support layer contained 1 mg of methyl paraoxybenzoate and the drug layer contained 1 mg of ethyl paraoxybenzoate. The dissolution rate of methyl paraoxybenzoate from the support layer and that of ethyl paraoxybenzoate from the drug layer of the preparation obtained above were measured by the following method. These dissolution rates were regarded as those of the support layer and the drug layer and the ratios of these dissolution rates were obtained.

TABLE 15
Formulation for dissolution test:
amount (g) per each layer of 100 g

| Name of ingredient | | Amount per 100 g |
|---|---|---|
| Support | HPC | 55.27 |
| layer | HPMCP | 23.64 |
| | Macrogol 400 | 9.09 |
| | Titanium oxide | 1.82 |
| | Sodium hydroxide | 1.09 |
| | Methyl paraoxybenzoate | 9.09 |
| Drug | Fentanyl citrate | 1.93 |
| layer | Titanium oxide | 1.95 |
| | Macrogol 400 | 9.76 |
| | Ethyl paraoxybenzoate | 2.44 |
| | HPC | 76.61 |
| | PVP K90 | 7.32 |

More specifically, using 500 mL of the second fluid of the dissolution test method defined in the Japanese Pharmacopeia as a test solution, a test was performed at a temperature of 37±0.5° C. at a paddle rotation number of 50 per minute in accordance with the U.S. Pharmacopeia <724> DRUG RELEASE Transdermal Delivery Systems-General Drug Release Standards (corresponding to the Japanese Pharmacopeia, the dissolution test method, Method 2, (paddle method)). The apparatus used in the test is the same as shown in FIGS. 3A and 3B. On the upper surface of the celluloid plate 13 of 80 mm×80 mm, which has the weight 15 adhered on the lower surface with a synthetic polymer adhesive agent, a rounded corner rectangular film preparation of 10 mm×12 mm (r=2.8 mm) was adhered by a double faced adhesive tape. In this way, the test patch was placed between the lower end of the paddle 12 and the bottom of the container 10.

The test was performed while stirring the test solution in the container by rotating the paddle. Every after 1, 3, 7 and 15 minutes, an aliquot of 2 mL was taken from the test solution in the container. Immediately after the aliquot was taken, 2 mL of fresh test solution warmed up to 37±0.5° C. was added to the container. After an aliquot was taken from the test solution 15 minutes after the initiation, the rotation number of the paddle was set at 150 per minute. When the preparation was completely dissolved, a 2 mL aliquot was taken from of the test solution, filtrated by a dissolution-test filter, and subjected to high performance liquid chromatography to measure the amounts of methyl paraoxybenzoate and ethyl paraoxybenzoate dissolved in each aliquot. The dissolution ratios of methyl paraoxybenzoate and ethyl paraoxybenzoate were calculated from the dissolution amounts of them at individual sampling time-points based on the dissolution amount of them when the preparation was completely dissolved being as 100%, and then, the dissolution rates of methyl paraoxybenzoate and ethyl paraoxybenzoate were obtained. Using the dissolution rates of methyl paraoxybenzoate and ethyl paraoxybenzoate as indexes of those of the support layer and drug layer, the dissolution-rate ratio of the drug layer to the support layer was obtained. The results of the dissolution test are shown in Table 16 and the graph of FIG. 7.

TABLE 16
Dissolution ratio of methyl paraoxybenzoate
and ethyl paraoxybenzoate (%)

| Time | Methyl | Ethyl | Dissolution-rate |
|---|---|---|---|
| (min.) | paraoxybenzoate | paraoxybenzoate | ratio (*) |
| 1 | 14.4 | 17.5 | 1.21 |
| 3 | 56.0 | 48.8 | 0.87 |
| 7 | 88.7 | 76.9 | 0.87 |
| 15 | 96.5 | 90.1 | 0.93 |

(*): (Ratio of Dissolution Rate = dissolution rate of drug layer/dissolution rate of support layer)

### Example 12

Using ingredients shown in Table 14, a disintegrating support layer solution and a disintegrating drug layer solution were obtained in the same manner as in Example 11.

Subsequently, the disintegrating support layer solution was spread on a polyester release liner and dried to obtain a support layer of 5 μm thick (intermediate product b). Separately, the disintegrating drug layer solution was spread on a polyester release liner and dried to obtain a drug layer of 45 μm thick (intermediate product c).

The intermediate products b and c were overlapped in such a manner that the support layer of intermediate product b is in contact with the drug layer of intermediate product c, and passed through a pair of lamination roll to laminate these intermediate products at a preparation temperature of 60° C., and a pressure of 0.3 MPa to obtain a double-layered film of 50 μm formed of the support layer and the drug layer. Of both polyester release liners attached to both sides, the film on the drug layer side was removed (intermediate product d)

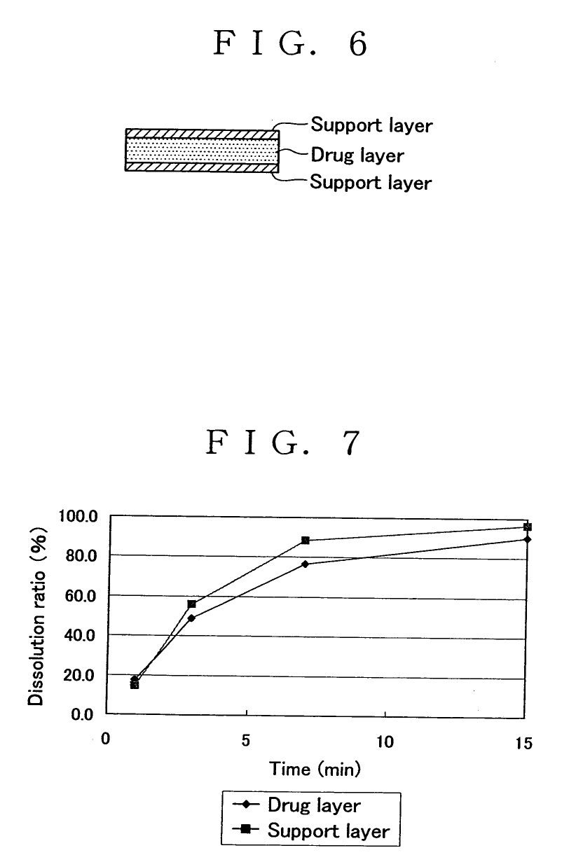

The intermediate product d with the polyester release liner was cut in half along its longitudinal direction to obtain two narrower halves. These two intermediate products d were overlapped in such a manner that the drug layers are in contact with each other, and passed through a pair of lamination rolls to laminate two intermediate products d at a preparation temperature of 60° C., and a pressure of 0.3 MPa to obtain a triple-layer film of 100 μm formed of support layer/drug layer/support layer, as shown in FIG. 6. Thereafter, if necessary, the release liner is removed from either one or both sides of the resultant laminated or multilayered film. The resultant film was punched into rectangular pieces of 10 mm×12 mm with rounded corners (r=2.8 mm) to obtain film preparations.

A pH value of the film preparation was measured in the same manner as in Example 11. The pH was 5.07. Furthermore, dissolution or disintegration states of the drug layer and the support layer were checked by the second fluid of the dissolution test method defined in the Japanese pharmacopeias. As a result, both the drug layer and the support layer were completely dissolved one hour after the initiation of stirring by the paddle, similarly as in Example 11.

### Comparative Example 11

Using the ingredients of Comparative Example 11 shown in FIG. 14, a film preparation was obtained in the same manner as in Example 11. Comparative Example 11 differs from Example 11 in that sodium hydroxide serving as (E) a pH-adjusting agent is contained in a drug layer but not in a support layer. The percent by weight of sodium hydroxide in the support layer of Example 11 is larger than that in the drug layer in Comparative Example 11. This is because the support layer is thinner than the drug layer. More specifically, since the weight of the support layer is lower than the drug layer, the percentage value increases due to the difference in weight. The absolute weight of sodium hydroxide contained in a preparation formed of the support layer and the drug layer is the same in Example 11 and Comparative Example 11.

### Comparison Between Examples 11 and 12 and Comparative Example 11

In an edible oral mucosal patch formed of a disintegrating support layer containing (A) a semi-synthetic water-insoluble polymer compound, (B) a semi-synthetic water-soluble polymer compound and (D) a water-soluble polyhydric alcohol; and a disintegrating drug layer containing a fentanyl compound, (B) a semi-synthetic water-soluble polymer compound, (C) a synthetic water-soluble polymer compound and (D) a water-soluble polyhydric alcohol, if an appropriate amount of (E) a pH-adjusting agent is contained not in the drug layer but in the disintegrating support layer, the stability of a main ingredient, a fentanyl compound, contained in the edible oral mucosal patch can be greatly improved without decreasing a transmucosal absorption efficiency of the fentanyl compound. To demonstrate this, the following comparative test was performed.

### [Stability Test]

In an edible oral mucosal patch formed of a disintegrating support layer and a disintegrating drug layer, the stability of a fentanyl compound may differ between the cases where (E) a pH-adjusting agent is contained in the disintegrating support layer and in the disintegrating drug layer. To confirm the effect of (E) a pH-adjusting agent upon the stability between the two cases, preparations of Examples 11 and 12 and Comparative Example 11 were subjected to the following stability test.

### Test Method:

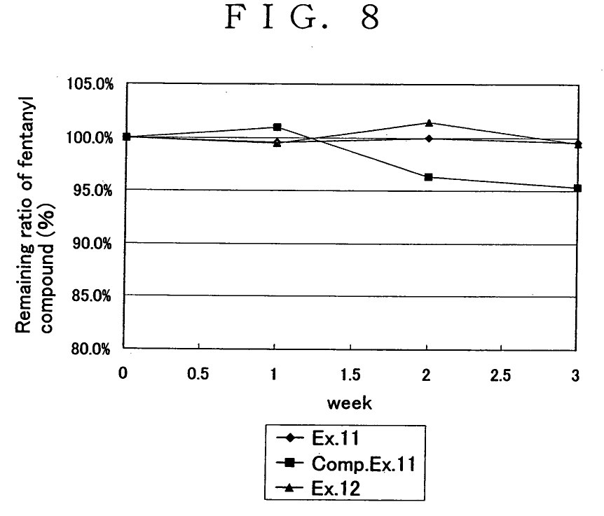

Film preparation samples were individually wrapped with a wrapping material formed of aluminum foil coated with polyacrylonitrile-based sealant and stored at 60° C. for 3 weeks (equivalent to the storage at room temperature for about three years). After the storage, a quality change with time was evaluated by subjecting samples to methanol extraction and high performance liquid chromatography to quantify fentanyl citrate contained in methanol. The remaining ratios of fentanyl citrate in preparations were calculated. The results are shown in FIG. 8

From FIG. 8, it is found that, in an edible oral mucosal patch formed of a disintegrating support layer containing (A) a semi-synthetic water-insoluble polymer compound, (B) a semi-synthetic water-soluble polymer compound and (D) a water-soluble polyhydric alcohol; and a disintegrating drug layer containing a fentanyl compound, (B) a semi-synthetic water-soluble polymer compound, (C) a synthetic water-soluble polymer compound and (D) a water-soluble polyhydric alcohol, the stability of a main ingredient, a fentanyl compound, can be greatly improved by adding (E) a pH-adjusting agent not in the drug layer but in the disintegrating support layer.

When Example 11 is compared to Example 12 in the graph of FIG. 8, the preparation of Example 12 is more stable. This is because the boundary between a support layer and a drug layer is more sharply distinguishable in Example 12, in which the support layer and the drug layer are multilayered by lamination, than in Example 11, in which the support layer and the drug layer are multilayered by coating, and therefore, the support layer and the drug layer are not partially mixed with each other in a preparation process. To describe more specifically, when sections of the edible oral mucosal patches of the Examples 11 and 12 are observed by a digital microscope BS-D8000II (trade name, manufactured by Sonic Co., Ltd.), the boundary between the support layer and the drug layer is sharply distinctive in Example 12 but indistinctive in Example 11. The boundary cannot be distinguished in Example 11. The reason is considered as follows: Since the drug layer solution is spread on the support layer which has been formed by coating process in Example 11, the drug layer solution spread on the support layer permeates into the underlying support layer. At that time, a fentanyl compound is migrated together with the solvent into the underlying support layer. The fentanyl compound migrated into the support layer is affected by the pH-adjusting agent contained in the support layer, degrading the stability of the fentanyl compound. Hence, the stability of a preparation can be more improved by physically laminating the support layer and the drug layer.

### [Transmucosal Permeability Test: Hamster Cheek Pouch In Vitro Test]

In an edible oral mucosal patch formed of a disintegrating support layer and a disintegrating drug layer, the case where (E) a pH-adjusting agent is added in the disintegrating support layer can exhibit the same transmucosal absorption efficiency of a main agent, a fentanyl compound, as the case where (E) a pH-adjusting agent is added in the disintegrating drug layer. To demonstrate this, the preparations of Examples 11 and 12 and Comparative Example 11 were subjected to the following transmucosal permeability test.

### Test Method:

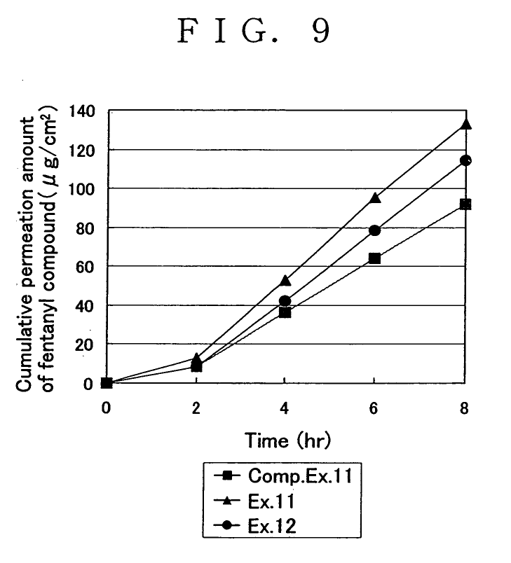

The cumulative permeation amount of a fentanyl compound passed through the cheek pouch mucosa was calculated in the same manner as the above-described transmucosal permeability test (e) using a hamster cheek pouch. The samples of the internal liquid were taken out after 2, 4, 6 and 8 hours. The results are shown in Table 17 and FIG. 9.

TABLE 17
Cumulative permeation amount of fentanyl compound (μg/cm²)

| | Time (hr) | | | | |
|---|---|---|---|---|---|
| Formulation No. | 0 | 2 | 4 | 6 | 8 |
| Example 11 | 0 | 13.1 | 53.0 | 95.4 | 133.3 |
| Example 12 | 0 | 8.5 | 42.0 | 78.6 | 114.5 |
| Comparative Example | 0 | 8.7 | 36.5 | 64.2 | 92.2 |
| 11 | | | | | |

As is apparent from the results of Table 17 and FIG. 9, in an edible oral mucosal patch formed of a disintegrating support layer containing (A) a semi-synthetic water-insoluble polymer compound, (B) a semi-synthetic water-soluble polymer compound and (D) a water-soluble polyhydric alcohol; and a disintegrating drug layer containing a fentanyl compound, (B) a semi-synthetic water-soluble polymer compound, (C) a synthetic water-soluble polymer compound and (D) a water-soluble polyhydric alcohol, the transmucosal permeability of a fentanyl compound in the case where (E) a pH-adjusting agent is added to the disintegrating support layer is the same as that in the case where (E) a pH-adjusting agent is added to the disintegrating drug layer. Therefore, it is found that the transmucosal absorption efficiency is not affected no matter which layer (E) a pH-adjusting agent is added.

As is apparent from the foregoing, according to the present invention, a fentanyl compound-containing edible patch to be applied to oral mucosa capable of attaching to the oral mucosa securely in an appropriate time and sufficiently absorbing a main ingredient, a fentanyl compound, through the mucosa, can be obtained for the first time by using all 4 types of ingredients: a semi-synthetic water-insoluble polymer compound, a semi-synthetic water-soluble polymer compound, a synthetic water-soluble polymer compound and a water-soluble polyhydric alcohol in combination, and further using a pH-adjusting agent.

In addition, the present invention can provide a fentanyl compound-containing edible patch to be applied to oral mucosa, which is industrially and efficiently produced, consists essentially of substances accepted as food additives or pharmaceutical additives approved for oral administration, and overcomes all problems: “punching crack” and “delamination” that should not occur in a manufacturing step; “adhesion to wrapping material”, “crack and/or fracture” and “decomposition of a fentanyl compound” that should not occur in a delivery/carrying step; and “poor adhesion”, “poor transmucosal permeability”, “early-stage dissolution” and “delamination” that should not occur during use, by using 5 types of ingredients: a semi-synthetic water-insoluble polymer compound, a semi-synthetic water-soluble polymer compound, a synthetic water-soluble polymer compound, a water-soluble polyhydric alcohol and a pH-adjusting agent in combination.

In particular, when the edible oral mucosal patch of the present invention is formed of a disintegrating support layer and a disintegration drug layer, if a pH-adjusting agent is contained not in the drug layer containing a main ingredient, a fentanyl compound, but in the support layer, a fentanyl compound can be absorbed more effectively through the mucosa and the stability of the fentanyl compound in the edible oral mucosal patch can be remarkably improved. The stability is further improved by multilayering the support layer and the drug layer by physical lamination rather than multilayering these layers by coating, since the boundary between the support layer and the drug layer is distinguishable and the layers are not partially mixed during a manufacturing process.

1-6. (canceled)

7. An edible oral mucosal patch, which comprises a disintegrating support layer containing a semi-synthetic water-insoluble polymer compound, a semi-synthetic water-soluble polymer compound, a water-soluble polyhydric alcohol and a pH-adjusting agent; and a disintegrating drug layer containing a fentanyl compound, a semi-synthetic water-soluble polymer compound, a synthetic water-soluble polymer compound and a water-soluble polyhydric alcohol.

8. The edible oral mucosal patch according to claim 7, wherein a dissolution-rate ratio of the drug layer to the support layer in the second fluid of the disintegration test method defined in the Japanese Pharmacopoeia falls within the range of 5:1 to 1:5.

9. The edible oral mucosal patch according to claim 7, wherein a dissolution-rate ratio of the drug layer to the support layer in the second fluid of the disintegration test method defined in the Japanese Pharmacopoeia falls within the range of 3:1 to 1:3.

10. The edible oral mucosal patch according to claim 7, wherein the support layer and the drug layer are multilayered by lamination.

11. The edible oral mucosal patch according to claim 7, wherein the fentanyl compound is fentanyl citrate.

12. The edible oral mucosal patch according to claim 7, wherein
the pH of an aqueous solution prepared by mixing 2 parts by weight of the oral mucosal patch with 98 parts by weight of water, followed by dissolving at least the drug layer is 4.0 to 8.0.

13. (canceled)
